(19) United States
(12) Patent Application Publication
(10) Pub. No.: US 2014/0155287 A1
(43) Pub. Date: Jun. 5, 2014

(54) METHODS AND COMPOSITIONS FOR ASSESSMENT OF PULMONARY FUNCTION AND DISORDERS

(71) Applicant: Robert Peter YOUNG, Auckland (NZ)

(72) Inventor: Robert Peter YOUNG, Auckland (NZ)

(73) Assignee: SYNERGENZ BIOSCIENCE LTD, HONG KONG (CN)

(21) Appl. No.: 14/154,012

(22) Filed: Jan. 13, 2014

Related U.S. Application Data
(63) Continuation of application No. 13/379,269, filed on Mar. 19, 2012, now abandoned.

(30) Foreign Application Priority Data
Jun. 19, 2009 (NZ) 577874
Apr. 29, 2010 (NZ) 584983

(51) Int. Cl. C12Q 1/68

(52) U.S. Cl. CPC C12Q 1/6886
USPC 506/9; 435/6.11

(57) ABSTRACT

The present invention provides methods for the assessment of risk of developing lung cancer in smokers and non-smokers using analysis of genetic polymorphisms. The present invention also relates to the use of genetic polymorphisms in assessing a subject's risk of developing lung cancer, and the suitability of a subject for an intervention in respect of lung cancer. Nucleotide probes and primers, kits, and microarrays suitable for such assessment are also provided.

### CROSS-REFERENCES

The present application is a Continuation application of U.S. Ser. No. 13/379,269, filed on Mar. 19, 2012, which is a U.S. National Stage application filed under 35 U.S.C. §371 of International Patent Application no. PCT/NZ2010/000117, filed on Jun. 18, 2010, designating the U.S., and published in English on Dec. 23, 2010, as International Patent Application Publication no. WO/2010/147489, which in turn claims priority to New Zealand Patent Application no. 577874 filed on Jun. 19, 2009, and New Zealand Patent Application no. 584983, filed on Apr. 29, 2010, each of the foregoing which is incorporated herein by reference in its entirety.

### FIELD OF THE INVENTION

The present invention is concerned with methods for assessment of pulmonary function and/or disorders, and in particular for assessing risk of developing lung cancer in smokers and non-smokers using analysis of genetic polymorphisms.

### BACKGROUND OF THE INVENTION

Lung cancer is the second most common cancer and has been attributed primarily to cigarette smoking. Other factors contributing to the development of lung cancer include occupational exposure, genetic factors, radon exposure, exposure to other aero-pollutants and possibly dietary factors (Alberg A J, et al., 2003). Non-smokers are estimated to have a one in 400 risk of lung cancer (0.25%). Smoking increases this risk by approximately 40 fold, such that smokers have a one in 10 risk of lung cancer (10%) and in long-term smokers the life-time risk of lung cancer has been reported to be as high 10-15% (Schwartz A G. 2004). Genetic factors are thought to play some part as evidenced by a weak familial tendency (among smokers) and the fact that only the minority of smokers get lung cancer. It is generally accepted that the majority of this genetic tendency comes from low penetrant high frequency polymorphisms, that is, polymorphisms which are common in the general population that in context of chronic smoking exposure contribute collectively to cancer development (Schwartz A G. 2004, Wu X et al., 2004). Several epidemiological studies have reported that impaired lung function (Anthonisen N R. 1989, Skillrud D M. 1986, Tockman M S et al., 1987, Kuller L H, et al., 1990, Nomura A, et al., 1991) or symptoms of obstructive lung disease (Mayne S T, et al., 1999) are independent risk factors for lung cancer and are possibly more relevant than smoking exposure dose.

Despite advances in the treatment of airways disease, current therapies do not significantly alter the natural history of lung cancer, which may include metastasis and progressive loss of lung function causing respiratory failure and death. Although cessation of smoking may be expected to reduce this decline in lung function, it is probable that if this is not achieved at an early stage, the loss is considerable and symptoms of worsening breathlessness likely cannot be averted. Analogous to the discovery of serum cholesterol and its link to coronary artery disease, there is a need to better understand the factors that contribute to lung cancer so that tests that identify at risk subjects can be developed and that new treatments can be discovered to reduce the adverse effects of lung cancer. The early diagnosis of lung cancer or of a propensity to developing lung cancer enables a broader range of prophylactic or therapeutic treatments to be employed than can be employed in the treatment of late stage lung cancer. Such prophylactic or early therapeutic treatment is also more likely to be successful, achieve remission, improve quality of life, and/or increase lifespan.

To date, a number of biomarkers useful in the diagnosis and assessment of propensity towards developing various pulmonary disorders have been identified. These include, for example, single nucleotide polymorphisms including the following: A−82G in the promoter of the gene encoding human macrophage elastase (MMP12); T→C within codon 10 of the gene encoding transforming growth factor beta (TGFβ); C+760G of the gene encoding superoxide dismutase 3 (SOD3); T−1296C within the promoter of the gene encoding tissue inhibitor of metalloproteinase 3 (TIMP3); and polymorphisms in linkage disequilibrium with these polymorphisms, as disclosed in PCT International Application PCT/NZ02/00106 (published as WO 02/099134 and incorporated herein by reference in its entirety).

It would be desirable and advantageous to have additional biomarkers which could be used to assess a subject's risk of developing pulmonary disorders such as lung cancer, or a risk of developing lung cancer-related impaired lung function, particularly if the subject is a smoker.

It is primarily to such biomarkers and their use in methods to assess risk of developing such disorders that the present invention is directed.

### SUMMARY OF THE INVENTION

The present invention is primarily based on the finding that certain polymorphisms are found more often in subjects with lung cancer than in control subjects. Analysis of these polymorphisms reveals an association between polymorphisms and the subject's risk of developing lung cancer.

Thus, according to one aspect there is provided a method of determining a subject's risk of developing lung cancer comprising analysing a sample from said subject for the presence or absence of one or more polymorphisms selected from the group consisting of:
rs1489759 A/G in the gene encoding Hedgehog Interacting Protein (HHIP);
rs2240997 G/A in the gene encoding Solute Carrier Family 34 (SLC34A2);
rs7671167 T/C in the Family with sequence similarity 13A (FAM13A) gene;
rs161974 C/T in gene encoding Bicaudal D homologue 1 (BICD1);
rs2630578 C/G in gene encoding BICD1;

wherein the presence or absence of said polymorphism is indicative of the subject's risk of developing lung cancer.

This polymorphism can be detected directly or by detection of one or more polymorphisms which are in linkage disequilibrium with one or more of said polymorphisms.

Linkage disequilibrium (LD) is a phenomenon in genetics whereby two or more mutations or polymorphisms are in such close genetic proximity that they are co-inherited. This means that in genotyping, detection of one polymorphism as present infers the presence of the other. (Reich D E et al; Linkage disequilibrium in the human genome, Nature 2001, 411:199-204.)

The lung cancer may be non-small cell lung cancer including adenocarcinoma and squamous cell carcinoma, or small cell lung cancer, or may be a carcinoid tumor, a lymphoma, or a metastatic cancer.

The method can additionally comprise analysing a sample from said subject for the presence or absence of one or more further polymorphisms selected from the group consisting of:
rs16969968 G/A in the gene encoding Nicotinic Acetylcholine receptor subunit alpha 3/5 (nAChR);
rs1051730 C/T in the gene encoding nAChR;
rs2202507 A/C in the gene encoding Glycophorin A Precursor Gene (GYPA);
rs1052486 A/G in the gene encoding HLA-B associated transcript 3 (BAT3);
rs2808630 T/C in the gene encoding C reactive protein (CRP);
rs401681 A/G in the cisplatin-resistance regulated gene 9 (CRR9) gene;
rs402710 A/G in the CRR9 gene;
rs1422795 T/C in the A Disintegrin and Metalloproteinase 19 (ADAM19) gene.

The method can further comprise analysing a sample from said subject for the presence or absence of one or more further polymorphisms selected from the group consisting of:
Ser307Ser G/T (rs1056503) in the X-ray repair complementing defective repair in Chinese hamster cells 4 gene (XRCC4);
A/T c74delA in the gene encoding cytochrome P450 polypeptide CYP3A43 (CYP3A43);
A/C (rs2279115) in the gene encoding B-cell CLL/lymphoma 2 (BCL2);
A/G at +3100 in the 3′UTR (rs2317676) of the gene encoding Integrin beta 3 (ITGB3);
−3714 G/T (rs6413429) in the gene encoding Dopamine transporter 1 (DAT1);
A/G (rs 1139417) in the gene encoding Tumor necrosis factor receptor 1 (TNFR1);
C/Del (rs1799732) in the gene encoding Dopamine receptor D2 (DRD2);
C/T (rs763110) in the gene encoding Fas ligand (FasL);
C/T (rs5743836) in the gene encoding Toll-like receptor 9 (TLR9);
R19W A/G (rs10115703) in the gene encoding Cerberus 1 (Cer 1);
K3326X A/T (rs11571833) in the breast cancer 2 early onset gene (BRCA2);
V433M A/G (rs2306022) in the gene encoding Integrin alpha-11;
E375G T/C (rs7214723) in the gene encoding Calcium/calmodulin-dependent protein kinase kinase 1 (CAMKK1);
−81 C/T (rs 2273953) in the 5′ UTR of the gene encoding Tumor protein P73 (P73);
Asp 298 Glu in the gene encoding Nitric oxide synthase 3 (NOS3);
−786 T/C in the promoter of the gene encoding Nitric oxide synthase 3;
Arg 312 Gln in the gene encoding Superoxide dismutase 3 (SOD3);
Ala 15 Thr in the gene encoding Anti-chymotrypsin (ACT);
Asn 357 Ser A/G in the gene encoding Matrix metalloproteinase 12 (MMP12);
105 A/C in the gene encoding Interleukin-18 (IL-18);
−133 G/C in the promoter of the gene encoding Interleukin-18;
874 A/T in the gene encoding Interferon gamma (IFNγ);
−765 G/C in the gene encoding Cyclooxygenase 2 (COX2);
−447 G/C in the gene encoding Connective tissue growth factor (CTGF);
−221 C/T in the gene encoding Mucin 5AC (MUC5AC);
+161 G/A in the gene encoding Mannose binding lectin 2 (MBL2);
intron 1 C/T in the gene encoding Arginase 1 (Arg 1);
Leu 252 Val C/G in the gene encoding Insulin-like growth factor II receptor (IGF2R);
−1082 A/G in the gene encoding Interleukin 10 (IL-10)
Arg 399 Gln G/A in the X-ray repair complementing defective in Chinese hamster 1 (XRCC1) gene;
−251 A/T in the gene encoding Interleukin-8 (IL-8);
A870G in the gene encoding Cyclin D (CCND1);
−511 A/G in the gene encoding Interleukin 1B (IL-1B);
−670G in the gene encoding FAS (Apo-1/CD95);
−751 G/T in the promoter of the Xeroderma pigmentosum complementation group D (XPD) gene;
Ile 462 Val A/G in the gene encoding Cytochrome P450 1A1 (CYP1A1);
Ser 326 Cys G/C in the gene encoding 8-Oxoguanine DNA glycolase (OGG1);
Arg 197 Gln A/G in the gene encoding N-acetyltransferase 2 (NAT2);
1019 G/C Pst I in the gene encoding Cytochrome P450 2E1 (CYP2E1);
C/T Rsa I in the gene encoding Cytochrome P450 2E1;
GSTM null in the gene encoding Glutathione S-transferase M (GSTM);
−1607 1G/2G in the promoter of the gene encoding Matrix metalloproteinase 1 (MMP1);
Gln 185 Glu G/C in the gene encoding Nibrin (NBS1);
Phe 257 Ser C/T in the gene encoding REV 1;
Asp 148 Glu G/T in the gene encoding Apex nuclease (APE1).

Again, detection of the one or more further polymorphisms may be carried out directly or by detection of polymorphisms in linkage disequilibrium with the one or more further polymorphisms.

The presence of one or more polymorphisms selected from the group consisting of:
the GG genotype at the rs1489759 A/G polymorphism in the gene encoding HHIP;
the CC genotype at the rs7671167 T/C polymorphism in the FAM13A gene;
the T allele at the rs161974 C/T polymorphism in the gene encoding BICD1;
the CC genotype at the rs2202507 A/C polymorphism in the gene encoding GYPA;
the CC genotype at the rs2808630 T/C polymorphism in the gene encoding CRP;
the TT genotype or the T allele at the rs7214723 E375G T/C polymorphism the gene encoding CAMKK1;
the CC genotype or the C allele at the −81 C/T (rs 2273953) polymorphism the gene encoding P73;
the AA genotype or the A allele at the A/C (rs2279115) polymorphism in the gene encoding BCL2;
the AG or GG genotype or the G allele at the +3100 A/G (rs2317676) polymorphism in the gene encoding ITGB3;
the CDel or DelDel genotype or the Del allele at the C/Del (rs1799732) polymorphism in the gene encoding DRD2;
the TT genotype at the C/T (rs763110) polymorphism the gene encoding FasL;
the TT genotype at the rs1799983 Asp 298 Glu polymorphism in the gene encoding NOS3;
the CG or GG genotype at the Arg 312 Gln polymorphism in the gene encoding SOD3;
the AG or GG genotype at the rs652438 Asn 357 Ser polymorphism in the gene encoding MMP 12;
the AC or CC genotype at the rs549908 105 A/C polymorphism in the gene encoding IL-18;
the AC or CC genotype at the rs549908 105 G/C polymorphism in the gene encoding IL-18;
the CC or CG genotype at the rs20417−765 G/C polymorphism in the promoter of the gene encoding COX2;
the TT genotype at the −221 C/T polymorphism in the gene encoding MUC5AC;
the TT genotype at the rs2781667 intron 1 C/T polymorphism in the gene encoding Arg1;
the GG genotype at the rs8191754 Leu252Val polymorphism in the gene encoding IGF2R;
the GG genotype at the rs1800896−1082 A/G polymorphism in the gene encoding IL-10;
the AA genotype at the rs4073−251 A/T polymorphism in the gene encoding IL-8;
the AA genotype at the rs25487 Arg 399 Gln polymorphism in the XRCC1 gene;
the GG genotype at the rs603965 A870G polymorphism in the gene encoding CCND1;
the GG genotype at the rs 13181−751 polymorphism in the promoter of the XPD gene;
the AG or GG genotype at the rs1048943 Ile 462 Val polymorphism in the gene encoding CYP1A1;
the GG genotype at the rs1052133 Ser 326 Cys polymorphism in the gene encoding OGG1; or
the CC genotype at the rs3087386 Phe 257 Ser polymorphism in the gene encoding REV1.
   may be indicative of a reduced risk of developing lung cancer.

The presence of one or more polymorphisms selected from the group consisting of:
the GA or AA genotype or the A allele at the rs2240997 G/A polymorphism in the gene encoding SLC34A2;
the C allele at the rs161974 C/T polymorphism in the gene encoding BICD1;
the CC genotype at the rs2630578 polymorphism in the gene encoding BICD1;
the AA genotype or the A allele at the rs16969968 G/A polymorphism in the gene encoding nAChR;
the TT genotype or the A allele at the rs1051730 C/T polymorphism in the gene encoding nAChR;
the GG genotype or the G allele at the rs1052486 A/G polymorphism in the gene encoding BAT3;
the GG genotype at the rs401681 A/G polymorphism in the CRR9 gene;
the GG genotype at the rs402710 A/G polymorphism in the CRR9 gene;
the CC genotype at the rs1422795 T/C polymorphism in the ADAM19 gene;
the AA or AG genotype or the A allele at the rs 10115703 R19W A/G polymorphism in the gene encoding Cer 1;
the GG or GT genotype or the G allele at the Ser307Ser G/T polymorphism in the XRCC4 gene;
the AT or TT genotype or the T allele at the K3326X A/T polymorphism in the BRCA2 gene;
the AA genotype or the A allele at the V433M A/G polymorphism in the gene encoding Integrin alpha-11;
the AT or TT genotype or the T allele at the A/T c74delA polymorphism in the gene encoding CYP3A43;
the GT or TT genotype at the −3714 G/T (rs6413429) polymorphism in the gene encoding DAT1;
the AA genotype or the A allele at the A/G (rs1139417) polymorphism in the gene encoding TNFR1;
the CC genotype at the C/T (rs5743836) polymorphism in the gene encoding TLR9;
the TT genotype at the rs2070744−786 T/C polymorphism in the promoter of the gene encoding NOS3;
the GG genotype at the rs4934 Ala 15 Thr polymorphism in the gene encoding ACT;
the AA genotype at the rs549908 105 A/C polymorphism in the gene encoding IL-18;
the CC genotype at the rs360721−133 G/C polymorphism in the promoter of the gene encoding IL-18;
the AA genotype at the rs2430561 874 A/T polymorphism in the gene encoding IFNγ;
the GG genotype at the rs20417−765 G/C polymorphism in the promoter of the gene encoding COX2;
the CC or GC genotype at the −447 G/C polymorphism in the gene encoding CTGF;
the AA or AG genotype at the rs1800450+161 G/A polymorphism in the gene encoding MBL2;
the GG genotype at the rs16944−511 A/G polymorphism in the gene encoding IL-1B;
the AA genotype at the rs1800682 A-670G polymorphism in the gene encoding FAS;
the GG genotype at the rs1799930 Arg 197 Gln polymorphism in the gene encoding NAT2;
the AA genotype at the rs1048943 Ile462 Val polymorphism in the gene encoding CYP 1A1;
the CC or CG genotype at the rs3813867 1019 G/C Pst I polymorphism in the gene encoding CYP2E1;
the TT or TC genotype at the rs2031920 C/T Rsa I polymorphism in the gene encoding CYP2E1;
the null genotype at the GSTM polymorphism in the gene encoding GSTM;
the 2G/2G genotype at the rs1799750−1607 1G/2G polymorphism in the promoter of the gene encoding MMP1;
the CC genotype at the rs1805794 Gln 185 Glu polymorphism in the gene encoding NBS1; or
the GG genotype at the rs3136820 Asp 148 Glu polymorphism in the gene encoding APE1 may be indicative of an increased risk of developing lung cancer.
The methods of the invention are particularly useful in smokers (both current and former).

It will be appreciated that the methods of the invention identify two categories of polymorphisms—namely those associated with a reduced risk of developing lung cancer (which can be termed “protective polymorphisms”) and those associated with an increased risk of developing lung cancer (which can be termed “susceptibility polymorphisms”).

Therefore, the present invention further provides a method of assessing a subject's risk of developing lung cancer, said method comprising:

determining the presence or absence of at least one protective polymorphism associated with a reduced risk of developing lung cancer; and

in the absence of at least one protective polymorphism, determining the presence or absence of at least one susceptibility polymorphism associated with an increased risk of developing lung cancer;

wherein the presence of one or more of said protective polymorphisms is indicative of a reduced risk of developing lung cancer, and the absence of at least one protective polymorphism in combination with the presence of at least one susceptibility polymorphism is indicative of an increased risk of developing lung cancer.

In one embodiment, the at least one protective polymorphism is the GG genotype at the rs 1489759 A/G polymorphism in the gene encoding HHIP or one or more polymorphism in linkage disequilibrium with the GG genotype at the rs 1489759 A/G polymorphism in the gene encoding HHIP.

In one embodiment, the at least one protective polymorphism is the CC genotype at the rs7671167 T/C polymorphism in the FAM13A gene or one or more polymorphism in linkage disequilibrium with the CC genotype at the rs7671167 T/C polymorphism in the FAM13A gene.

In one embodiment, the at least one susceptibility polymorphism is the GA or AA genotype or the A allele at the rs2240997 G/A polymorphism in the gene encoding SLC34A2, or one or more polymorphisms in linkage disequilibrium with the GA or AA genotype or the A allele at the rs2240997 G/A polymorphism in the gene encoding SLC34A2.

In other embodiments, the at least one protective polymorphism or the at least one susceptibility polymorphism is selected from the groups defined above.

In a preferred form of the invention the presence of two or more protective polymorphisms is indicative of a reduced risk of developing lung cancer.

In a further preferred form of the invention the presence of two or more susceptibility polymorphisms is indicative of an increased risk of developing lung cancer.

In still a further preferred form of the invention the presence of two or more protective polymorphisms irrespective of the presence of one or more susceptibility polymorphisms is indicative of reduced risk of developing lung cancer.

In another aspect, the invention provides a method of determining a subject's risk of developing lung cancer, said method comprising providing the result of one or more genetic tests of a sample from said subject, and analysing the result for the presence or absence of one or more polymorphisms selected from the group consisting of:
rs1489759 A/G in the gene encoding Hedgehog Interacting Protein (HHIP);
rs2240997 G/A in the gene encoding Solute Carrier Family 34 (SLC34A2);
rs7671167 T/C in the Family with sequence similarity 13A (FAM13A) gene;
rs161974 C/T in the gene encoding BICD1;
rs2630578 C/G in the gene encoding BICD1;
or one or more polymorphisms in linkage disequilibrium with one or more of these polymorphisms;

wherein a result indicating the presence or absence of one or more of said polymorphisms is indicative of the subject's risk of developing lung cancer.

The method can additionally comprise providing the result of one or more genetic tests of a sample from said subject, and analysing the result for the presence or absence of one or more further polymorphisms selected from the group consisting of:
rs16969968 G/A in the gene encoding Nicotinic Acetylcholine receptor subunit alpha 3/5 (nAChR);
rs1051730 C/T in the gene encoding nAChR;
rs2202507 A/C in the gene encoding Glycophorin A Precursor Gene (GYPA);
rs1052486 A/G in the gene encoding HLA-B associated transcript 3 (BAT3);
rs2808630 T/C in the gene encoding C reactive protein (CRP);
rs401681 A/G in the cisplatin-resistance regulated gene 9 (CRR9) gene;
rs402710 A/G in the CRR9 gene;
rs1422795 T/C in the A Disintegrin and Metalloproteinase 19 (ADAM19) gene.

Again, the presence or absence may be determined directly or by determining the presence or absence of polymorphisms in linkage disequilibrium with the one or more further polymorphisms.

In a further aspect there is provided a method of determining a subject's risk of developing lung cancer comprising the analysis of two or more polymorphisms selected from the groups defined above.

In one embodiment of the methods and uses of the present invention one or more of the following polymorphisms are selected:
rs1489759 A/G in the gene encoding HHIP;
rs2240997 G/A in the gene encoding SLC34A2;
rs7671167 T/C in the Family with sequence similarity 13A (FAM13A) gene;
rs161974 C/T in the gene encoding BICD1;
rs2630578 C/G in the gene encoding BICD1;
rs16969968 G/A in the gene encoding nAChR;
rs1051730 C/T in the gene encoding nAChR;
rs2202507 A/C in the gene encoding GYPA;
rs1052486 A/G in the gene encoding BAT3;
rs2808630 T/C in the gene encoding CRP;
rs401681 A/G in the cisplatin-resistance regulated gene 9 (CRR9) gene;
rs402710 A/G in the CRR9 gene;
rs1422795 T/C/ in the A Disintegrin and Metalloproteinase 19 (ADAM19) gene;
or one or more polymorphisms in linkage disequilibrium with any one or more of these polymorphisms;
and each of the following polymorphisms are selected:
−133 G/C (rs360721) in the promoter of the gene encoding Interleukin-18;
−251 A/T (rs4073) in the gene encoding Interleukin-8;
Arg 197 Gln (rs 1799930) in the gene encoding N-acetylcysteine transferase 2;
Ala 15 Thr A/G (rs4934) in the gene encoding α1-antichymotrypsin;
−3714 G/T (rs6413429) in the gene encoding DAT1;
−81 C/T (rs 2273953) in the 5′ UTR of the gene encoding P73;
Arg 312 Gln (rs1799895) in the gene encoding SOD3;
A/G at +3100 in the 3′UTR (rs2317676) of the gene encoding ITGB3;
C/Del (rs1799732) in the gene encoding DRD2;

or one or more polymorphisms in linkage disequilibrium with any one or more of these polymorphisms.

In one embodiment of the methods and uses of the present invention one or more of the following polymorphisms are selected:
rs1489759 A/G in the gene encoding HHIP;
rs2240997 G/A in the gene encoding SLC34A2;
rs7671167 T/C in the Family with sequence similarity 13A (FAM13A) gene;
rs161974 C/T in the gene encoding BICD1;
rs2630578 C/G in the gene encoding BICD1;
rs16969968 G/A in the gene encoding nAChR;
rs1051730 C/T in the gene encoding nAChR;
rs2202507 A/C in the gene encoding GYPA;
rs1052486 A/G in the gene encoding BAT3;
rs2808630 T/C in the gene encoding CRP;
   or one or more polymorphisms in linkage disequilibrium with any one or more of these polymorphisms;
and each of the following polymorphisms are selected:
−133 G/C (rs360721) in the promoter of the gene encoding Interleukin-18;
−251 A/T (rs4073) in the gene encoding Interleukin-8;
Arg 197 Gln (rs 1799930) in the gene encoding N-acetylcysteine transferase 2;
Ala 15 Thr A/G (rs4934) in the gene encoding α1-antichymotrypsin;
−3714 G/T (rs6413429) in the gene encoding DAT1;
−81 C/T (rs 2273953) in the 5′ UTR of the gene encoding P73;
Arg 312 Gln (rs1799895) in the gene encoding SOD3;
A/G at +3100 in the 3′UTR (rs2317676) of the gene encoding ITGB3;
C/Del (rs1799732) in the gene encoding DRD2;
or one or more polymorphisms in linkage disequilibrium with any one or more of these polymorphisms.

In one embodiment of the methods and uses of the present invention one or more of the following polymorphisms are selected:
rs1489759 A/G in the gene encoding HHIP;
rs2240997 G/A in the gene encoding SLC34A2;
rs7671167 T/C in the Family with sequence similarity 13A (FAM13A) gene;
rs161974 C/T in the gene encoding BICD1;
rs2630578 C/G in the gene encoding BICD1;
rs16969968 G/A in the gene encoding nAChR;
rs1051730 C/T in the gene encoding nAChR;
rs2202507 A/C in the gene encoding GYPA;
rs1052486 A/G in the gene encoding BAT3;
rs2808630 T/C in the gene encoding CRP;
rs401681 A/G in the cisplatin-resistance regulated gene 9 (CRR9) gene;
rs402710 A/G in the CRR9 gene;
rs1422795 T/C/ in the A Disintegrin and Metalloproteinase 19 (ADAM19) gene;
or one or more polymorphisms in linkage disequilibrium with any one or more of these polymorphisms;
and each of the following polymorphisms are selected:
−133 G/C (rs360721) in the promoter of the gene encoding Interleukin-18;
−251 A/T (rs4073) in the gene encoding Interleukin-8;
Arg 197 Gln (rs 1799930) in the gene encoding N-acetylcysteine transferase 2;
Ala 15 Thr A/G (rs4934) in the gene encoding α1-antichymotrypsin;
−3714 G/T (rs6413429) in the gene encoding DAT1;
−81 C/T (rs 2273953) in the 5′ UTR of the gene encoding P73;
Arg 312 Gln (rs1799895) in the gene encoding SOD3;
A/G at +3100 in the 3′UTR (rs2317676) of the gene encoding ITGB3;
C/Del (rs1799732) in the gene encoding DRD2;
A/C (rs2279115) in the gene encoding BCL2;
or one or more polymorphisms in linkage disequilibrium with any one or more of these polymorphisms.

In one embodiment of the methods and uses of the present invention one or more of the following polymorphisms are selected:
rs1489759 A/G in the gene encoding HHIP;
rs2240997 G/A in the gene encoding SLC34A2;
rs7671167 T/C in the Family with sequence similarity 13A (FAM13A) gene;
rs161974 C/T in the gene encoding BICD1;
rs2630578 C/G in the gene encoding BICD1;
rs16969968 G/A in the gene encoding nAChR;
rs1051730 C/T in the gene encoding nAChR;
rs2202507 A/C in the gene encoding GYPA;
rs1052486 A/G in the gene encoding BAT3;
rs2808630 T/C in the gene encoding CRP;
rs401681 A/G in the cisplatin-resistance regulated gene 9 (CRR9) gene;
rs402710 A/G in the CRR9 gene;
rs1422795 T/C/ in the A Disintegrin and Metalloproteinase 19 (ADAM19) gene;
or one or more polymorphisms in linkage disequilibrium with any one or more of these polymorphisms;
and each of the following polymorphisms are selected:
−133 G/C (rs360721) in the promoter of the gene encoding Interleukin-18;
−251 A/T (rs4073) in the gene encoding Interleukin-8;
Arg 197 Gln (rs 1799930) in the gene encoding N-acetylcysteine transferase 2;
Ala 15 Thr A/G (rs4934) in the gene encoding α1-antichymotrypsin;
−3714 G/T (rs6413429) in the gene encoding DAT1;
−81 C/T (rs 2273953) in the 5′ UTR of the gene encoding P73;
Arg 312 Gln (rs1799895) in the gene encoding SOD3;
A/G at +3100 in the 3′UTR (rs2317676) of the gene encoding ITGB3;
C/Del (rs1799732) in the gene encoding DRD2;
A/C (rs2279115) in the gene encoding BCL2;
V433M A/G (rs2306022) in the gene encoding ITGA11;
or one or more polymorphisms in linkage disequilibrium with any one or more of these polymorphisms.

In one embodiment of the methods and uses of the present invention one or more of the following polymorphisms are selected:
rs1489759 A/G in the gene encoding HHIP;
rs2240997 G/A in the gene encoding SLC34A2;
rs7671167 T/C in the Family with sequence similarity 13A (FAM13A) gene;
rs161974 C/T in the gene encoding BICD1;
rs2630578 C/G in the gene encoding BICD1;
rs16969968 G/A in the gene encoding nAChR;
rs1051730 C/T in the gene encoding nAChR;
rs2202507 A/C in the gene encoding GYPA;
rs1052486 A/G in the gene encoding BAT3;
rs2808630 T/C in the gene encoding CRP;
rs401681 A/G in the cisplatin-resistance regulated gene 9 (CRR9) gene;
rs402710 A/G in the CRR9 gene;
rs1422795 T/C/ in the A Disintegrin and Metalloproteinase 19 (ADAM19) gene;
or one or more polymorphisms in linkage disequilibrium with any one or more of these polymorphisms;
and each of the following polymorphisms are selected:
Rsa 1 C/T (rs2031920) in the gene encoding CYP 2E1;
−133 G/C (rs360721) in the promoter of the gene encoding Interleukin-18;
−251 A/T (rs4073) in the gene encoding Interleukin-8;
−511 A/G (rs 16944) in the gene encoding Interleukin 1B;
V433M A/G (rs2306022) in the gene encoding ITGA11;
Arg 197 Gln A/G (rs 1799930) in the gene encoding N-acetylcysteine transferase 2;
Ala 15 Thr A/G (rs4934) in the gene encoding α1-antichymotrypsin;
R19W A/G (rs 10115703) in the gene encoding Cerberus 1;
−3714 G/T (rs6413429) in the gene encoding DAT1;
A/G (rs1139417) in the gene encoding TNFR1;
C/T (rs5743836) in the gene encoding TLR9;
−81 C/T (rs 2273953) in the 5′ UTR of the gene encoding P73;
Arg 312 Gln (rs1799895) in the gene encoding SOD3;
A/G at +3100 in the 3′UTR (rs2317676) of the gene encoding ITGB3;
C/Del (rs1799732) in the gene encoding DRD2;
A/C (rs2279115) in the gene encoding BCL2;
−751 G/T (rs 13181) in the promoter of the gene encoding XPD;
Phe 257 Ser C/T (rs3087386) in the gene encoding REV1;
C/T (rs763110) in the gene encoding FasL;
or one or more polymorphisms in linkage disequilibrium with any one or more of these polymorphisms.

In a preferred form of the invention the methods as described herein are performed in conjunction with an analysis of one or more risk factors, including one or more epidemiological risk factors, associated with a risk of developing lung cancer. Such epidemiological risk factors include but are not limited to smoking or exposure to tobacco smoke, age, sex, and familial history of lung cancer.

In another aspect the invention provides a set of nucleotide probes and/or primers for use in the preferred methods of the invention herein described. Preferably, the nucleotide probes and/or primers are those which span, or are able to be used to span, the polymorphic regions of the genes. Also provided are one or more nucleotide probes and/or primers comprising the sequence of any one of the probes and/or primers herein described, including any one comprising or consisting of the sequence of any 12 or more contiguous nucleotides from one of SEQ.ID.NO. 1 to 9. In yet a further aspect, the invention provides a nucleic acid microarray for use in the methods of the invention, which microarray comprises a substrate presenting nucleic acid sequences capable of hybridizing to nucleic acid sequences which encode one or more of the susceptibility or protective polymorphisms described herein or sequences complimentary thereto.

In another aspect, the invention provides an antibody microarray for use in the methods of the invention. In one embodiment the microarray comprises a substrate presenting antibodies capable of binding to a product of expression of a gene the expression of which is upregulated or downregulated when associated with a susceptibility or protective polymorphism as described herein. In another embodiment, the microarray comprises a substrate presenting one or more antibodies capable of binding to a gene product of one of the polymorphic genes described herein. Particularly contemplated are antibodies capable of discriminating between a gene product encoded by a gene comprising one or other of the alleles at a polymorphic site, including one or more antibodies capable of binding (including improved binding) a gene product encoded by one allelic form of a polymorphic gene. For example, where one allele of a polymorphism elicits an amino acid substitution in the encoded protein, a suitable antibody may preferentially bind the protein gene product comprising an amino acid substitution encoded by one of the alleles at a polymorphic site.

It will be appreciated that such antibodies may be useful in the methods of the invention in embodiments not relying on microarrays, and may instead comprise a kit as described herein, optionally together with one or more other reagents, instructions for use, and the like.

In a further aspect the present invention provides a method treating a subject having an increased risk of developing lung cancer comprising the step of replicating, genotypically or phenotypically, the presence and/or functional effect of a protective polymorphism in said subject.

In yet a further aspect, the present invention provides a method of treating a subject having an increased risk of developing lung cancer, said subject having a detectable susceptibility polymorphism which either upregulates or downregulates expression of a gene such that the physiologically active concentration of the expressed gene product is outside a range which is normal for the age and sex of the subject, said method comprising the step of restoring the physiologically active concentration of said product of gene expression to be within a range which is normal for the age and sex of the subject.

In yet a further aspect, the present invention provides a method for screening for compounds that modulate the expression and/or activity of a gene, the expression of which is upregulated or downregulated when associated with a susceptibility or protective polymorphism, said method comprising the steps of:

contacting a candidate compound with a cell comprising a susceptibility or protective polymorphism which has been determined to be associated with the upregulation or downregulation of expression of a gene; and

measuring the expression of said gene following contact with said candidate compound,

wherein a change in the level of expression after the contacting step as compared to before the contacting step is indicative of the ability of the compound to modulate the expression and/or activity of said gene.

Preferably, said cell is a human lung cell which has been pre-screened to confirm the presence of said polymorphism.

Preferably, said cell comprises a susceptibility polymorphism associated with upregulation of expression of said gene and said screening is for candidate compounds which downregulate expression of said gene.

Alternatively, said cell comprises a susceptibility polymorphism associated with downregulation of expression of said gene and said screening is for candidate compounds which upregulate expression of said gene.

In another embodiment, said cell comprises a protective polymorphism associated with upregulation of expression of said gene and said screening is for candidate compounds which further upregulate expression of said gene.

Alternatively, said cell comprises a protective polymorphism associated with downregulation of expression of said gene and said screening is for candidate compounds which further downregulate expression of said gene.

In another aspect, the present invention provides a method for screening for compounds that modulate the expression and/or activity of a gene, the expression of which is upregulated or downregulated when associated with a susceptibility or protective polymorphism, said method comprising the steps of:

contacting a candidate compound with a cell comprising a gene, the expression of which is upregulated or downregulated when associated with a susceptibility or protective polymorphism but which in said cell the expression of which is neither upregulated nor downregulated; and

measuring the expression of said gene following contact with said candidate compound,

wherein a change in the level of expression after the contacting step as compared to before the contacting step is indicative of the ability of the compound to modulate the expression and/or activity of said gene.

Preferably, expression of the gene is downregulated when associated with a susceptibility polymorphism and said screening is for candidate compounds which in said cell, upregulate expression of said gene.

Preferably, said cell is a human lung cell which has been pre-screened to confirm the presence, and baseline level of expression, of said gene.

Alternatively, expression of the gene is upregulated when associated with a susceptibility polymorphism and said screening is for candidate compounds which, in said cell, downregulate expression of said gene.

In another embodiment, expression of the gene is upregulated when associated with a protective polymorphism and said screening is for compounds which, in said cell, upregulate expression of said gene.

Alternatively, expression of the gene is downregulated when associated with a protective polymorphism and said screening is for compounds which, in said cell, downregulate expression of said gene.

In yet a further aspect, the present invention provides a method of assessing the likely responsiveness of a subject at risk of developing or suffering from lung cancer to a prophylactic or therapeutic treatment, which treatment involves restoring the physiologically active concentration of a product of gene expression to be within a range which is normal for the age and sex of the subject, which method comprises detecting in said subject the presence or absence of a susceptibility polymorphism which when present either upregulates or downregulates expression of said gene such that the physiological active concentration of the expressed gene product is outside said normal range, wherein the detection of the presence of said polymorphism is indicative of the subject likely responding to said treatment.

In still a further aspect, the present invention provides a method of assessing a subject's suitability for an intervention that is diagnostic of or therapeutic for a disease, the method comprising:

a) providing a net score for said subject, wherein the net score is or has been determined by:
i) providing the result of one or more genetic tests of a sample from the subject, and analysing the result for the presence or absence of protective polymorphisms and for the presence or absence of susceptibility polymorphisms, wherein said protective and susceptibility polymorphisms are associated with said disease,
ii) assigning a positive score for each protective polymorphism and a negative score for each susceptibility polymorphism or vice versa;
iii) calculating a net score for said subject by representing the balance between the combined value of the protective polymorphisms and the combined value of the susceptibility polymorphisms present in the subject sample; and

b) providing a distribution of net scores for disease sufferers and non-sufferers wherein the net scores for disease sufferers and non-sufferers are or have been determined in the same manner as the net score determined for said subject;

c) determining whether the net score for said subject lies within a threshold on said distribution separating individuals deemed suitable for said intervention from those for whom said intervention is deemed unsuitable;

wherein a net score within said threshold is indicative of the subject's suitability for the intervention, and wherein a net score outside the threshold is indicative of the subject's unsuitability for the intervention.

The value assigned to each protective polymorphism may be the same or may be different. The value assigned to each susceptibility polymorphism may be the same or may be different, with either each protective polymorphism having a negative value and each susceptibility polymorphism having a positive value, or vice versa.

In one embodiment, the intervention is a diagnostic test for said disease.

In another embodiment, the intervention is a therapy for said disease, more preferably a preventative therapy for said disease.

Preferably, the disease is lung cancer, more preferably the disease is lung cancer and the protective and susceptibility polymorphisms are selected from the group consisting of:
rs1489759 A/G in the gene encoding Hedgehog Interacting Protein (HHIP);
rs2240997 G/A in the gene encoding Solute Carrier Family 34 (SLC34A2);
rs7671167 T/C in the Family with sequence similarity 13A (FAM13A) gene;
rs161974 C/T in the gene encoding BICD1;
rs2630578 C/G in the gene encoding BICD1;
rs16969968 G/A in the gene encoding Nicotinic Acetylcholine receptor subunit alpha 3/5 (nAChR);
rs1051730 C/T in the gene encoding nAChR;
rs2202507 A/C in the gene encoding Glycophorin A Precursor Gene (GYPA);
rs1052486 A/G in the gene encoding HLA-B associated transcript 3 (BAT3);
rs2808630 T/C in the gene encoding C reactive protein (CRP);
rs401681 A/G in the cisplatin-resistance regulated gene 9 (CRR9) gene;
rs402710 A/G in the CRR9 gene;
rs1422795 T/C/ in the A Disintegrin and Metalloproteinase 19 (ADAM19) gene.

In another embodiment, the present invention provides a kit for assessing a subject's risk of developing one or more obstructive lung diseases selected from lung cancer, said kit comprising a reagent for analysing a sample from said subject for the presence or absence of one or more polymorphisms described herein.

Particularly contemplated are kits comprising a reagent for analysing a sample from said subject for the presence or absence of one or more polymorphisms selected from the group consisting of:
rs1489759 A/G in the gene encoding Hedgehog Interacting Protein (HHIP);
rs2240997 G/A in the gene encoding Solute Carrier Family 34 (SLC34A2);
rs7671167 T/C in the Family with sequence similarity 13A (FAM13A) gene;
rs161974 C/T in the gene encoding BICD1;
rs2630578 C/G in the gene encoding BICD1;
or one or more polymorphisms which are in linkage disequilibrium with one or more of these polymorphisms.

The term “comprising” as used in this specification means “consisting at least in part of”. When interpreting each statement in this specification that includes the term “comprising”, features other than that or those prefaced by the term may also be present. Related terms such as “comprise” and “comprises” are to be interpreted in the same manner.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

### BRIEF DESCRIPTION OF FIGURES

FIG. 1: depicts a graph showing polymorphisms in linkage disequilibrium with the nAChR polymorphisms specified herein.

FIG. 2: depicts a graph showing the cumulative effect of the 9 SNP panel of protective and susceptible SNPs in combination with non-genetic variables to derive a lung cancer risk score in lung cancer cases and controls.

### DESCRIPTION OF THE PREFERRED EMBODIMENTS

Using case-control studies the frequencies of several genetic variants (polymorphisms) of candidate genes in smokers who have developed lung cancer and blood donor controls have been compared. The majority of these candidate genes have confirmed (or likely) functional effects on gene expression or protein function. Specifically the frequencies of polymorphisms between blood donor controls, resistant smokers and those with lung cancer (subdivided into those with early onset and those with normal onset) have been compared. The present invention demonstrates that there are both protective and susceptibility polymorphisms present in selected candidate genes of the patients tested.

In one embodiment described herein 9 susceptibility genetic polymorphisms and 5 protective genetic polymorphism are identified. These are as follows:

| | Genotype | | | |
|---|---|---|---|---|
| Gene and SNP | Allele | Phenotype | OR | P value |
| HHIP rs1489759 A/G | GG | protective | 0.7 | 0.05 |
| SLC34A2; rs2240997 G/A | GA/AA | susceptiblility | 1.53 | 0.009 |
| | A | susceptibility | 1.4 | 0.01 |
| FAM13A; rs7671167 T/C | CC | protective | 0.71 | 0.02 |
| nAChR; rs16969968 G/A | AA | susceptiblility | 1.8 | 0.005 |
| | A | susceptiblility | 1.4 | 0.001 |
| nAChR; rs1051730 C/T | TT | susceptiblility | 1.9 | 0.002 |
| | T | susceptiblility | 1.4 | 0.005 |
| GYPA; rs2202507 A/C | CC | protective | 0.70 | 0.02 |
| BAT3 rs1052486 A/G | GG | susceptiblility | 1.4 | 0.08 |
| | G | susceptiblility | 1.2 | 0.07 |
| CRP rs2808630 T/C | CC | protective | 0.68 | 0.09 |
| CRR9 rs401681 A/G | GG | susceptibility | 1.4 | 0.05 |
| CRR9 rs402710 A/G | GG | susceptibility | 1.4 | 0.05 |
| ADAM19 rs1422795 T/C | CC | susceptibility | 1.41 | 0.10 |
| BICD1 rs161974 C/T | C | susceptibility | 1.24 | 0.022 |
| | T | protective | | |
| BICD1 rs2630578 C/G | CC | susceptibility | 1.8 | 0.067 |

A susceptibility genetic polymorphism is one which, when present, is indicative of an increased risk of developing lung cancer. In contrast, a protective genetic polymorphism is one which, when present, is indicative of a reduced risk of developing lung cancer.

As used herein, the phrase “risk of developing lung cancer” means the likelihood that a subject to whom the risk applies will develop lung cancer, and includes predisposition to, and potential onset of the disease. Accordingly, the phrase “increased risk of developing lung cancer” means that a subject having such an increased risk possesses an hereditary inclination or tendency to develop lung cancer. This does not mean that such a person will actually develop lung cancer at any time, merely that he or she has a greater likelihood of developing lung cancer compared to the general population of individuals that either does not possess a polymorphism associated with increased lung cancer or does possess a polymorphism associated with decreased lung cancer risk. Subjects with an increased risk of developing lung cancer include those with a predisposition to lung cancer, such as a tendency or predilection regardless of their lung function at the time of assessment, for example, a subject who is genetically inclined to lung cancer but who has normal lung function, those at potential risk, including subjects with a tendency to mildly reduced lung function who are likely to go on to suffer lung cancer if they keep smoking, and subjects with potential onset of lung cancer, who have a tendency to poor lung function on spirometry etc., consistent with lung cancer at the time of assessment.

Similarly, the phrase “decreased risk of developing lung cancer” means that a subject having such a decreased risk possesses an hereditary disinclination or reduced tendency to develop lung cancer. This does not mean that such a person will not develop lung cancer at any time, merely that he or she has a decreased likelihood of developing lung cancer compared to the general population of individuals that either does possess one or more polymorphisms associated with increased lung cancer, or does not possess a polymorphism associated with decreased lung cancer.

It will be understood that in the context of the present invention the term “polymorphism” means the occurrence together in the same population at a rate greater than that attributable to random mutation (usually greater than 1%) of two or more alternate forms (such as alleles or genetic markers) of a chromosomal locus that differ in nucleotide sequence or have variable numbers of repeated nucleotide units. See www.ornl.gov/sci/techresources/Human_Genome/publicat/97pr/09 gloss.html#p. Accordingly, the term “polymorphisms” is used herein contemplates genetic variations, including single nucleotide substitutions, insertions and deletions of nucleotides, repetitive sequences (such as microsatellites), and the total or partial absence of genes (e.g. null mutations). As used herein, the term “polymorphisms” also includes genotypes and haplotypes. A genotype is the genetic composition at a specific locus or set of loci. A haplotype is a set of closely linked genetic markers present on one chromosome which are not easily separable by recombination, tend to be inherited together, and may be in linkage disequilibrium. A haplotype can be identified by patterns of polymorphisms such as SNPs. Similarly, the term “single nucleotide polymorphism” or “SNP” in the context of the present invention includes single base nucleotide substitutions and short deletion and insertion polymorphisms.

As used herein, the phrase “presence or absence of a polymorphism” and grammatical equivalents includes the presence or absence of one or other of the alleles at the polymorphism.

A reduced or increased risk of a subject developing lung cancer may be diagnosed by analysing a sample from said subject for the presence of a polymorphism selected from the group consisting of:
rs1489759 A/G in the gene encoding Hedgehog Interacting Protein (HHIP);
rs2240997 G/A in the gene encoding Solute Carrier Family 34 (SLC34A2);
rs7671167 T/C in the Family with sequence similarity 13A (FAM13A) gene;
rs16969968 G/A in the gene encoding Nicotinic Acetylcholine receptor subunit alpha 3/5 (nAChR);
rs1051730 C/T in the gene encoding nAChR;
rs2202507 A/C in the gene encoding Glycophorin A Precursor Gene (GYPA);
rs1052486 A/G in the gene encoding HLA-B associated transcript 3 (BAT3);
rs2808630 T/C in the gene encoding C reactive protein (CRP);
or one or more polymorphisms which are in linkage disequilibrium with any one or more of the above group.

These polymorphisms can also be analysed in combinations of two or more, or in combination with other polymorphisms indicative of a subject's risk of developing lung cancer inclusive of the remaining polymorphisms listed above.

Expressly contemplated are combinations of the above polymorphisms with polymorphisms as described in PCT International application PCT/NZ02/00106, published as WO 02/099134, or the polymorphisms as described in PCT International application PCT/NZ2006/000125, published as WO2006/123955, or those polymorphisms described in PCT/NZ2007/000310, published as WO 2008/048120.

In one embodiment of the methods and uses of the present invention one or more of the following polymorphisms are selected:
rs1489759 A/G in the gene encoding HHIP;
rs2240997 G/A in the gene encoding SLC34A2;
rs7671167 T/C in the Family with sequence similarity 13A (FAM13A) gene;
rs161974 C/T in the gene encoding BICD1;
rs2630578 C/G in the gene encoding BICD1;
rs16969968 G/A in the gene encoding nAChR;
rs1051730 C/T in the gene encoding nAChR;
rs2202507 A/C in the gene encoding GYPA;
rs1052486 A/G in the gene encoding BAT3;
rs2808630 T/C in the gene encoding CRP;
rs401681 A/G in the cisplatin-resistance regulated gene 9 (CRR9) gene;
rs402710 A/G in the CRR9 gene;
rs1422795 T/C/ in the A Disintegrin and Metalloproteinase 19 (ADAM19) gene;
or one or more polymorphisms in linkage disequilibrium with any one or more of these polymorphisms;
and each of the following polymorphisms are selected:
−133 G/C (rs360721) in the promoter of the gene encoding Interleukin-18;
−251 A/T (rs4073) in the gene encoding Interleukin-8;
Arg 197 Gln (rs 1799930) in the gene encoding N-acetylcysteine transferase 2;
Ala 15 Thr A/G (rs4934) in the gene encoding α1-antichymotrypsin;
−3714 G/T (rs6413429) in the gene encoding DAT1;
−81 C/T (rs 2273953) in the 5′ UTR of the gene encoding P73;
Arg 312 Gln (rs1799895) in the gene encoding SOD3;
A/G at +3100 in the 3′UTR (rs2317676) of the gene encoding ITGB3;
C/Del (rs1799732) in the gene encoding DRD2;
or one or more polymorphisms in linkage disequilibrium with any one or more of these polymorphisms.

In one embodiment of the methods and uses of the present invention one or more of the following polymorphisms are selected:
rs1489759 A/G in the gene encoding HHIP;
rs2240997 G/A in the gene encoding SLC34A2;
rs7671167 T/C in the Family with sequence similarity 13A (FAM13A) gene;
rs161974 C/T in the gene encoding BICD1;
rs2630578 C/G in the gene encoding BICD1;
rs16969968 G/A in the gene encoding nAChR;
rs1051730 C/T in the gene encoding nAChR;
rs2202507 A/C in the gene encoding GYPA;
rs1052486 A/G in the gene encoding BAT3;
rs2808630 T/C in the gene encoding CRP;
rs401681 A/G in the cisplatin-resistance regulated gene 9 (CRR9) gene;
rs402710 A/G in the CRR9 gene;
rs1422795 T/C/ in the A Disintegrin and Metalloproteinase 19 (ADAM19) gene;
or one or more polymorphisms in linkage disequilibrium with any one or more of these polymorphisms;
and each of the following polymorphisms are selected:
−133 G/C (rs360721) in the promoter of the gene encoding Interleukin-18;
−251 A/T (rs4073) in the gene encoding Interleukin-8;
Arg 197 Gln (rs 1799930) in the gene encoding N-acetylcysteine transferase 2;
Ala 15 Thr A/G (rs4934) in the gene encoding α1-antichymotrypsin;
−3714 G/T (rs6413429) in the gene encoding DAT1;
−81 C/T (rs 2273953) in the 5′ UTR of the gene encoding P73;
Arg 312 Gln (rs1799895) in the gene encoding SOD3;
A/G at +3100 in the 3′UTR (rs2317676) of the gene encoding ITGB3;
C/Del (rs1799732) in the gene encoding DRD2;
or one or more polymorphisms in linkage disequilibrium with any one or more of these polymorphisms.

In one embodiment of the methods and uses of the present invention one or more of the following polymorphisms are selected:
rs1489759 A/G in the gene encoding HHIP;
rs2240997 G/A in the gene encoding SLC34A2;
rs7671167 T/C in the Family with sequence similarity 13A (FAM13A) gene;
rs161974 C/T in the gene encoding BICD1;
rs2630578 C/G in the gene encoding BICD1;
rs16969968 G/A in the gene encoding nAChR;
rs1051730 C/T in the gene encoding nAChR;
rs2202507 A/C in the gene encoding GYPA;
rs1052486 A/G in the gene encoding BAT3;
rs2808630 T/C in the gene encoding CRP;
rs401681 A/G in the cisplatin-resistance regulated gene 9 (CRR9) gene;
rs402710 A/G in the CRR9 gene;
rs1422795 T/C/ in the A Disintegrin and Metalloproteinase 19 (ADAM19) gene;
or one or more polymorphisms in linkage disequilibrium with any one or more of these polymorphisms;
and each of the following polymorphisms are selected:
−133 G/C (rs360721) in the promoter of the gene encoding Interleukin-18;
−251 A/T (rs4073) in the gene encoding Interleukin-8;
Arg 197 Gln (rs 1799930) in the gene encoding N-acetylcysteine transferase 2;
Ala 15 Thr A/G (rs4934) in the gene encoding α1-antichymotrypsin;
−3714 G/T (rs6413429) in the gene encoding DAT1;
−81 C/T (rs 2273953) in the 5′ UTR of the gene encoding P73;
Arg 312 Gln (rs1799895) in the gene encoding SOD3;
A/G at +3100 in the 3′UTR (rs2317676) of the gene encoding ITGB3;
C/Del (rs1799732) in the gene encoding DRD2;
A/C (rs2279115) in the gene encoding BCL2;
or one or more polymorphisms in linkage disequilibrium with any one or more of these polymorphisms.

In one embodiment of the methods and uses of the present invention one or more of the following polymorphisms are selected:
rs1489759 A/G in the gene encoding HHIP;
rs2240997 G/A in the gene encoding SLC34A2;
rs7671167 T/C in the Family with sequence similarity 13A (FAM13A) gene;
rs161974 C/T in the gene encoding BICD1;
rs2630578 C/G in the gene encoding BICD1;
rs16969968 G/A in the gene encoding nAChR;
rs1051730 C/T in the gene encoding nAChR;
rs2202507 A/C in the gene encoding GYPA;
rs1052486 A/G in the gene encoding BAT3;
rs2808630 T/C in the gene encoding CRP;
rs401681 A/G in the cisplatin-resistance regulated gene 9 (CRR9) gene;
rs1422795 T/C/ in the A Disintegrin and Metalloproteinase 19 (ADAM19) gene;
or one or more polymorphisms in linkage disequilibrium with any one or more of these polymorphisms;
and each of the following polymorphisms are selected:
−133 G/C (rs360721) in the promoter of the gene encoding Interleukin-18;
−251 A/T (rs4073) in the gene encoding Interleukin-8;
Arg 197 Gln (rs 1799930) in the gene encoding N-acetylcysteine transferase 2;
Ala 15 Thr A/G (rs4934) in the gene encoding α1-antichymotrypsin;
−3714 G/T (rs6413429) in the gene encoding DAT1;
−81 C/T (rs 2273953) in the 5′ UTR of the gene encoding P73;
Arg 312 Gln (rs1799895) in the gene encoding SOD3;
A/G at +3100 in the 3′UTR (rs2317676) of the gene encoding ITGB3;
C/Del (rs1799732) in the gene encoding DRD2;
A/C (rs2279115) in the gene encoding BCL2;
V433M A/G (rs2306022) in the gene encoding ITGA11;
or one or more polymorphisms in linkage disequilibrium with any one or more of these polymorphisms.

In one embodiment of the methods and uses of the present invention one or more of the following polymorphisms are selected:
rs1489759 A/G in the gene encoding HHIP;
rs2240997 G/A in the gene encoding SLC34A2;
rs7671167 T/C in the Family with sequence similarity 13A (FAM13A) gene;
rs161974 C/T in the gene encoding BICD1;
rs2630578 C/G in the gene encoding BICD1;
rs16969968 G/A in the gene encoding nAChR;
rs1051730 C/T in the gene encoding nAChR;
rs2202507 A/C in the gene encoding GYPA;
rs1052486 A/G in the gene encoding BAT3;
rs2808630 T/C in the gene encoding CRP;
rs401681 A/G in the cisplatin-resistance regulated gene 9 (CRR9) gene;
rs402710 A/G in the CRR9 gene;
rs1422795 T/C/ in the A Disintegrin and Metalloproteinase 19 (ADAM19) gene;
or one or more polymorphisms in linkage disequilibrium with any one or more of these polymorphisms;
and each of the following polymorphisms are selected:
Rsa 1 C/T (rs2031920) in the gene encoding CYP 2E1;
−133 G/C (rs360721) in the promoter of the gene encoding Interleukin-18;
−251 A/T (rs4073) in the gene encoding Interleukin-8;
−511 A/G (rs 16944) in the gene encoding Interleukin 1B;
V433M A/G (rs2306022) in the gene encoding ITGA11;
Arg 197 Gln A/G (rs 1799930) in the gene encoding N-acetylcysteine transferase 2;
Ala 15 Thr A/G (rs4934) in the gene encoding α1-antichymotrypsin;
R19W A/G (rs 10115703) in the gene encoding Cerberus 1;
−3714 G/T (rs6413429) in the gene encoding DAT1;
A/G (rs1139417) in the gene encoding TNFR1;
C/T (rs5743836) in the gene encoding TLR9;
−81 C/T (rs 2273953) in the 5′ UTR of the gene encoding P73;
Arg 312 Gln (rs1799895) in the gene encoding SOD3;
A/G at +3100 in the 3′UTR (rs2317676) of the gene encoding ITGB3;
C/Del (rs1799732) in the gene encoding DRD2;
A/C (rs2279115) in the gene encoding BCL2;
−751 G/T (rs 13181) in the promoter of the gene encoding XPD;
Phe 257 Ser C/T (rs3087386) in the gene encoding REV1;
C/T (rs763110) in the gene encoding FasL;
or one or more polymorphisms in linkage disequilibrium with any one or more of these polymorphisms.

Assays which involve combinations of polymorphisms, including those amenable to high throughput, such as those utilising Fast Real-Time PCR or mass spectrometry (such as that described herein in the Examples) or microarrays, are preferred.

Statistical analyses, particularly of the combined effects of these polymorphisms, show that the genetic analyses of the present invention can be used to determine the risk quotient of any smoker and in particular to identify smokers at greater risk of developing lung cancer. Such combined analysis can be of combinations of susceptibility polymorphisms only, of protective polymorphisms only, or of combinations of both. Analysis can also be step-wise, with analysis of the presence or absence of protective polymorphisms occurring first and then with analysis of susceptibility polymorphisms proceeding only where no protective polymorphisms are present.

Thus, through systematic analysis of the frequency of these polymorphisms in well defined groups of smokers and non-smokers, as described herein, it is possible to implicate certain proteins in the development of lung cancer and improve the ability to identify which smokers are at increased risk of developing lung cancer-related impaired lung function and lung cancer for predictive purposes.

The present results show for the first time that the minority of smokers who develop lung cancer do so because they have one or more of the susceptibility polymorphisms and few or none of the protective polymorphisms defined herein. It is thought that the presence of one or more susceptible polymorphisms, together with the damaging irritant and oxidant effects of smoking, combine to make this group of smokers highly susceptible to developing lung cancer. Additional risk factors, such as familial history, age, weight, pack years, etc., will also have an impact on the risk profile of a subject, and can be assessed in combination with the genetic analyses described herein.

The one or more polymorphisms can be detected directly or by detection of one or more polymorphisms which are in linkage disequilibrium with said one or more polymorphisms. As discussed above, linkage disequilibrium is a phenomenon in genetics whereby two or more mutations or polymorphisms are in such close genetic proximity that they are co-inherited. This means that in genotyping, detection of one polymorphism as present infers the presence of the other. (Reich D E et al; Linkage disequilibrium in the human genome, Nature 2001, 411:199-204.)

Various degrees of linkage disequilibrium are possible. Preferably, the one or more polymorphisms in linkage disequilibrium with one or more of the polymorphisms specified herein are in greater than about 60% linkage disequilibrium, are in about 70% linkage disequilibrium, about 75%, about 80%, about 85%, about 90%, about 91%, 92%, 93%, 94%, 95%, 96%, 97%, 98%, 99%, or about 100% linkage disequilibrium with one or more of the polymorphisms specified herein. Those skilled in the art will appreciate that linkage disequilibrium may also, when expressed with reference to the deviation of the observed frequency of a pair of alleles from the expected, be denoted by a capital D. Accordingly, the phrase “two alleles are in LD” usually means that D does not equal 0. Contrariwise, “linkage equilibrium” denotes the case D=0. When utilising this nomenclature, the one or more polymorphisms in LD with the one or more polymorphisms specified herein are preferably in LD of greater than about D′=0.6, of about D′=0.7, of about D′=0.75, of about D′=0.8, of about D′=0.85, of about D′=0.9, of about D′=0.91, of about D′=0.92, of about D′=0.93, of about D′=0.94, of about D′=0.95, of about D′=0.96, of about D′=0.97, of about D′=0.98, of about D′=0.99, or about D′=1.0. (Devlin and Risch 1995; A comparison of linkage disequilibrium measures for fine-scale mapping, Genomics 29: 311-322).

It will be apparent that polymorphisms in linkage disequilibrium with one or more other polymorphism associated with increased or decreased risk of developing lung cancer will also provide utility as biomarkers for risk of developing lung cancer. The data presented herein shows that the frequency for SNPs in linkage disequilibrium is very similar. Accordingly, these genetically linked SNPs can be utilized in combined polymorphism analyses to derive a level of risk comparable to that calculated from the original SNP.

It will therefore be apparent that one or more polymorphisms in linkage disequilibrium with the polymorphisms specified herein can be identified, for example, using public data bases. Examples of such polymorphisms reported to be in linkage disequilibrium with the polymorphisms specified herein are presented herein in Tables 18 to 24.

It will also be apparent that frequently a variety of nomenclatures may exist for any given polymorphism or for any given gene. For example, the gene referred to herein as the breast cancer 2 early onset gene is also variously referred to as BRCC2, Breast Cancer 2 Gene, Breast Cancer Type 2, Breast Cancer Type 2 Susceptibility Gene, Breast cancer type 2 susceptibility protein, FACD, FAD, FAD1, FANCB, FANCD1, and Hereditary Breast Cancer 2. When referring to a susceptibility or protective polymorphism as herein described, such alternative nomenclatures are also contemplated by the present invention.

The methods of the invention are primarily directed to the detection and identification of the above polymorphisms associated with lung cancer, which are all single nucleotide polymorphisms. In general terms, a single nucleotide polymorphism (SNP) is a single base change or point mutation resulting in genetic variation between individuals. SNPs occur in the human genome approximately once every 100 to 300 bases, and can occur in coding or non-coding regions. Due to the redundancy of the genetic code, a SNP in the coding region may or may not change the amino acid sequence of a protein product. A SNP in a non-coding region can, for example, alter gene expression by, for example, modifying control regions such as promoters, transcription factor binding sites, processing sites, ribosomal binding sites, and affect gene transcription, processing, and translation.

SNPs can facilitate large-scale association genetics studies, and there has recently been great interest in SNP discovery and detection. SNPs show great promise as markers for a number of phenotypic traits (including latent traits), such as for example, disease propensity and severity, wellness propensity, and drug responsiveness including, for example, susceptibility to adverse drug reactions. Knowledge of the association of a particular SNP with a phenotypic trait, coupled with the knowledge of whether an individual has said particular SNP, can enable the targeting of diagnostic, preventative and therapeutic applications to allow better disease management, to enhance understanding of disease states and to ultimately facilitate the discovery of more effective treatments, such as personalised treatment regimens.

Indeed, a number of databases have been constructed of known SNPs, and for some such SNPs, the biological effect associated with a SNP. For example, the NCBI SNP database “dbSNP” is incorporated into NCBI's Entrez system and can be queried using the same approach as the other Entrez databases such as PubMed and GenBank. This database has records for over 17 million SNPs mapped onto the human genome sequence. Each dbSNP entry includes the sequence context of the polymorphism (i.e., the surrounding sequence), the occurrence frequency of the polymorphism (by population or individual), and the experimental method(s), protocols, and conditions used to assay the variation, and can include information associating a SNP with a particular phenotypic trait.

At least in part because of the potential impact on health and wellness, there has been and continues to be a great deal of effort to develop methods that reliably and rapidly identify SNPs. This was no trivial task, at least in part because of the complexity of human genomic DNA, with a haploid genome of 3×10⁹ base pairs, and the associated sensitivity and discriminatory requirements.

Genotyping approaches to detect SNPs well-known in the art include DNA sequencing, methods that require allele specific hybridization of primers or probes, allele specific incorporation of nucleotides to primers bound close to or adjacent to the polymorphisms (often referred to as “single base extension”, or “minisequencing”), allele-specific ligation (joining) of oligonucleotides (ligation chain reaction or ligation padlock probes), allele-specific cleavage of oligonucleotides or PCR products by restriction enzymes (restriction fragment length polymorphisms analysis or RFLP) or chemical or other agents, resolution of allele-dependent differences in electrophoretic or chromatographic mobilities, by structure specific enzymes including invasive structure specific enzymes, or mass spectrometry. Analysis of amino acid variation is also possible where the SNP lies in a coding region and results in an amino acid change.

DNA sequencing allows the direct determination and identification of SNPs. The benefits in specificity and accuracy are generally outweighed for screening purposes by the difficulties inherent in whole genome, or even targeted subgenome, sequencing.

Mini-sequencing involves allowing a primer to hybridize to the DNA sequence adjacent to the SNP site on the test sample under investigation. The primer is extended by one nucleotide using all four differentially tagged fluorescent dideoxynucleotides (A, C, G, or T), and a DNA polymerase. Only one of the four nucleotides (homozygous case) or two of the four nucleotides (heterozygous case) is incorporated. The base that is incorporated is complementary to the nucleotide at the SNP position.

A number of sequencing methods and platforms are particularly suited to large-scale implementation, and are amenable to use in the methods of the invention. These include pyrosequencing methods, such as that utilised in the GS FLX pyrosequencing platform available from 454 Life Sciences (Branford, Conn.) which can generate 100 million nucleotide data in a 7.5 hour run with a single machine, and solid-state sequencing methods, such as that utilised in the SOLiD sequencing platform (Applied Biosystems, Foster City, Calif.).

A number of methods currently used for SNP detection involve site-specific and/or allele-specific hybridisation. These methods are largely reliant on the discriminatory binding of oligonucleotides to target sequences containing the SNP of interest. The techniques of Illumina (San Diego, Calif.), (Santa Clara, Calif.) and Nanogen Inc. (San Diego, Calif.) are particularly well-known, and utilize the fact that DNA duplexes containing single base mismatches are much less stable than duplexes that are perfectly base-paired. The presence of a matched duplex is usually detected by fluorescence. A number of whole-genome genotyping products and solutions amenable or adaptable for use in the present invention are now available, including those available from the above companies.

The majority of methods to detect or identify SNPs by site-specific hybridisation require target amplification by methods such as PCR to increase sensitivity and specificity (see, for example U.S. Pat. No. 5,679,524, PCT publication WO 98/59066, PCT publication WO 95/12607). US Patent Application publication number 20050059030 (incorporated herein by reference in its entirety) describes a method for detecting a single nucleotide polymorphism in total human DNA without prior amplification or complexity reduction to selectively enrich for the target sequence, and without the aid of any enzymatic reaction. The method utilises a single-step hybridization involving two hybridization events: hybridization of a first portion of the target sequence to a capture probe, and hybridization of a second portion of said target sequence to a detection probe. Both hybridization events happen in the same reaction, and the order in which hybridisation occurs is not critical.

US Patent Application publication number 20050042608 (incorporated herein by reference in its entirety) describes a modification of the method of electrochemical detection of nucleic acid hybridization of Thorp et al. (U.S. Pat. No. 5,871,918). Briefly, capture probes are designed, each of which has a different SNP base and a sequence of probe bases on each side of the SNP base. The probe bases are complementary to the corresponding target sequence adjacent to the SNP site. Each capture probe is immobilized on a different electrode having a non-conductive outer layer on a conductive working surface of a substrate. The extent of hybridization between each capture probe and the nucleic acid target is detected by detecting the oxidation-reduction reaction at each electrode, utilizing a transition metal complex. These differences in the oxidation rates at the different electrodes are used to determine whether the selected nucleic acid target has a single nucleotide polymorphism at the selected SNP site.

The technique of Lynx Therapeutics (Hayward, Calif.) using MEGATYPE™ technology can genotype very large numbers of SNPs simultaneously from small or large pools of genomic material. This technology uses fluorescently labeled probes and compares the collected genomes of two populations, enabling detection and recovery of DNA fragments spanning SNPs that distinguish the two populations, without requiring prior SNP mapping or knowledge.

A number of other methods for detecting and identifying SNPs exist. These include the use of mass spectrometry, for example, to measure probes that hybridize to the SNP. This technique varies in how rapidly it can be performed, from a few samples per day to a high throughput of many thousands of SNPs per day, using mass code tags. A preferred example is the use of mass spectrometric determination of a nucleic acid sequence which comprises the polymorphisms of the invention, for example, which includes the HHIP gene or a complementary sequence. Such mass spectrometric methods are known to those skilled in the art, and the genotyping methods of the invention are amenable to adaptation for the mass spectrometric detection of the polymorphisms of the invention, for example, by using the methods described in PCT/NZ2007/000310 published as WO 2008/048120.

SNPs can also be determined by ligation-bit analysis. This analysis requires two primers that hybridize to a target with a one nucleotide gap between the primers. Each of the four nucleotides is added to a separate reaction mixture containing DNA polymerase, ligase, target DNA and the primers. The polymerase adds a nucleotide to the 3′ end of the first primer that is complementary to the SNP, and the ligase then ligates the two adjacent primers together. Upon heating of the sample, if ligation has occurred, the now larger primer will remain hybridized and a signal, for example, fluorescence, can be detected. A further discussion of these methods can be found in U.S. Pat. Nos. 5,919,626; 5,945,283; 5,242,794; and 5,952,174.

U.S. Pat. No. 6,821,733 (incorporated herein by reference in its entirety) describes methods to detect differences in the sequence of two nucleic acid molecules that includes the steps of: contacting two nucleic acids under conditions that allow the formation of a four-way complex and branch migration; contacting the four-way complex with a tracer molecule and a detection molecule under conditions in which the detection molecule is capable of binding the tracer molecule or the four-way complex; and determining binding of the tracer molecule to the detection molecule before and after exposure to the four-way complex. Competition of the four-way complex with the tracer molecule for binding to the detection molecule indicates a difference between the two nucleic acids.

Protein- and proteomics-based approaches are also suitable for polymorphism detection and analysis. Polymorphisms which result in or are associated with variation in expressed proteins can be detected directly by analysing said proteins. This typically requires separation of the various proteins within a sample, by, for example, gel electrophoresis or HPLC, and identification of said proteins or peptides derived therefrom, for example by NMR or protein sequencing such as chemical sequencing or more prevalently mass spectrometry. Proteomic methodologies are well known in the art, and have great potential for automation. For example, integrated systems, such as the ProteomIQ™ system from Proteome Systems, provide high throughput platforms for proteome analysis combining sample preparation, protein separation, image acquisition and analysis, protein processing, mass spectrometry and bioinformatics technologies.

The majority of proteomic methods of protein identification utilise mass spectrometry, including ion trap mass spectrometry, liquid chromatography (LC) and LC/MSn mass spectrometry, gas chromatography (GC) mass spectroscopy, Fourier transform-ion cyclotron resonance-mass spectrometer (FT-MS), MALDI-TOF mass spectrometry, and ESI mass spectrometry, and their derivatives. Mass spectrometric methods are also useful in the determination of post-translational modification of proteins, such as phosphorylation or glycosylation, and thus have utility in determining polymorphisms that result in or are associated with variation in post-translational modifications of proteins.

Associated technologies are also well known, and include, for example, protein processing devices such as the “Chemical Inkjet Printer” comprising piezoelectric printing technology that allows in situ enzymatic or chemical digestion of protein samples electroblotted from 2-D PAGE gels to membranes by jetting the enzyme or chemical directly onto the selected protein spots. After in-situ digestion and incubation of the proteins, the membrane can be placed directly into the mass spectrometer for peptide analysis.

A large number of methods reliant on the conformational variability of nucleic acids have been developed to detect SNPs.

For example, Single Strand Conformational Polymorphism (SSCP, Orita et al., PNAS 1989 86:2766-2770) is a method reliant on the ability of single-stranded nucleic acids to form secondary structure in solution under certain conditions. The secondary structure depends on the base composition and can be altered by a single nucleotide substitution, causing differences in electrophoretic mobility under nondenaturing conditions. The various polymorphs are typically detected by autoradiography when radioactively labelled, by silver staining of bands, by hybridisation with detectably labelled probe fragments or the use of fluorescent PCR primers which are subsequently detected, for example by an automated DNA sequencer.

Modifications of SSCP are well known in the art, and include the use of differing gel running conditions, such as for example differing temperature, or the addition of additives, and different gel matrices. Other variations on SSCP are well known to the skilled artisan, including, RNA-SSCP, restriction endonuclease fingerprinting-SSCP, dideoxy fingerprinting (a hybrid between dideoxy sequencing and SSCP), bi-directional dideoxy fingerprinting (in which the dideoxy termination reaction is performed simultaneously with two opposing primers), and Fluorescent PCR-SSCP (in which PCR products are internally labelled with multiple fluorescent dyes, may be digested with restriction enzymes, followed by SSCP, and analysed on an automated DNA sequencer able to detect the fluorescent dyes).

Other methods which utilise the varying mobility of different nucleic acid structures include Denaturing Gradient Gel Electrophoresis (DGGE), Temperature Gradient Gel Electrophoresis (TGGE), and Heteroduplex Analysis (HET). Here, variation in the dissociation of double stranded DNA (for example, due to base-pair mismatches) results in a change in electrophoretic mobility. These mobility shifts are used to detect nucleotide variations.

Denaturing High Pressure Liquid Chromatography (HPLC) is yet a further method utilised to detect SNPs, using HPLC methods well-known in the art as an alternative to the separation methods described above (such as gel electophoresis) to detect, for example, homoduplexes and heteroduplexes which elute from the HPLC column at different rates, thereby enabling detection of mismatch nucleotides and thus SNPs.

Yet further methods to detect SNPs rely on the differing susceptibility of single stranded and double stranded nucleic acids to cleavage by various agents, including chemical cleavage agents and nucleolytic enzymes. For example, cleavage of mismatches within RNA:DNA heteroduplexes by RNase A, of heteroduplexes by, for example bacteriophage T4 endonuclease YII or T7 endonuclease I, of the 5′ end of the hairpin loops at the junction between single stranded and double stranded DNA by cleavase I, and the modification of mispaired nucleotides within heteroduplexes by chemical agents commonly used in Maxam-Gilbert sequencing chemistry, are all well known in the art.

Further examples include the Protein Translation Test (PTT), used to resolve stop codons generated by variations which lead to a premature termination of translation and to protein products of reduced size, and the use of mismatch binding proteins. Variations are detected by binding of, for example, the MutS protein, a component of Escherichia coli DNA mismatch repair system, or the human hMSH2 and GTBP proteins, to double stranded DNA heteroduplexes containing mismatched bases. DNA duplexes are then incubated with the mismatch binding protein, and variations are detected by mobility shift assay. For example, a simple assay is based on the fact that the binding of the mismatch binding protein to the heteroduplex protects the heteroduplex from exonuclease degradation.

Those skilled in the art will know that a particular SNP, particularly when it occurs in a regulatory region of a gene such as a promoter, can be associated with altered expression of a gene. Altered expression of a gene can also result when the SNP is located in the coding region of a protein-encoding gene, for example where the SNP is associated with codons of varying usage and thus with tRNAs of differing abundance. Such altered expression can be determined by methods well known in the art, and can thereby be employed to detect such SNPs. Similarly, where a SNP occurs in the coding region of a gene and results in a non-synonymous amino acid substitution, such substitution can result in a change in the function of the gene product. Similarly, in cases where the gene product is an RNA, such SNPs can result in a change of function in the RNA gene product. Any such change in function, for example as assessed in an activity or functionality assay, can be employed to detect such SNPs.

The above methods of detecting and identifying SNPs are amenable to use in the methods of the invention.

Of course, in order to detect and identify SNPs in accordance with the invention, a sample containing material to be tested is obtained from the subject. The sample can be any sample potentially containing the target SNPs (or target polypeptides, as the case may be) and obtained from any bodily fluid (blood, urine, saliva, etc) biopsies or other tissue preparations.

DNA or RNA can be isolated from the sample according to any of a number of methods well known in the art. For example, methods of purification of nucleic acids are described in Tijssen; Laboratory Techniques in Biochemistry and Molecular Biology: Hybridization with nucleic acid probes Part 1: Theory and Nucleic acid preparation, Elsevier, New York, N.Y. 1993, as well as in Maniatis, T., Fritsch, E. F. and Sambrook, J., Molecular Cloning Manual 1989.

To assist with detecting the presence or absence of polymorphisms/SNPs, nucleic acid probes and/or primers can be provided. Such probes have nucleic acid sequences specific for chromosomal changes evidencing the presence or absence of the polymorphism and are preferably labeled with a substance that emits a detectable signal when combined with the target polymorphism.

The nucleic acid probes can be genomic DNA or cDNA or mRNA, or any RNA-like or DNA-like material, such as peptide nucleic acids, branched DNAs, and the like. The probes can be sense or antisense polynucleotide probes. Where target polynucleotides are double-stranded, the probes may be either sense or antisense strands. Where the target polynucleotides are single-stranded, the probes are complementary single strands.

The probes can be prepared by a variety of synthetic or enzymatic schemes, which are well known in the art. The probes can be synthesized, in whole or in part, using chemical methods well known in the art (Caruthers et al., Nucleic Acids Res., Symp. Ser., 215-233 (1980)). Alternatively, the probes can be generated, in whole or in part, enzymatically.

Nucleotide analogs can be incorporated into probes by methods well known in the art. The only requirement is that the incorporated nucleotide analog must serve to base pair with target polynucleotide sequences. For example, certain guanine nucleotides can be substituted with hypoxanthine, which base pairs with cytosine residues. However, these base pairs are less stable than those between guanine and cytosine. Alternatively, adenine nucleotides can be substituted with 2,6-diaminopurine, which can form stronger base pairs than those between adenine and thymidine.

Additionally, the probes can include nucleotides that have been derivatized chemically or enzymatically. Typical chemical modifications include derivatization with acyl, alkyl, aryl or amino groups.

The probes can be immobilized on a substrate. Preferred substrates are any suitable rigid or semi-rigid support including membranes, filters, chips, slides, wafers, fibers, magnetic or nonmagnetic beads, gels, tubing, plates, polymers, microparticles and capillaries. The substrate can have a variety of surface forms, such as wells, trenches, pins, channels and pores, to which the polynucleotide probes are bound. Preferably, the substrates are optically transparent.

Furthermore, the probes do not have to be directly bound to the substrate, but rather can be bound to the substrate through a linker group. The linker groups are typically about 6 to 50 atoms long to provide exposure to the attached probe. Preferred linker groups include ethylene glycol oligomers, diamines, diacids and the like. Reactive groups on the substrate surface react with one of the terminal portions of the linker to bind the linker to the substrate. The other terminal portion of the linker is then functionalized for binding the probe.

The probes can be attached to a substrate by dispensing reagents for probe synthesis on the substrate surface or by dispensing preformed DNA fragments or clones on the substrate surface. Typical dispensers include a micropipette delivering solution to the substrate with a robotic system to control the position of the micropipette with respect to the substrate. There can be a multiplicity of dispensers so that reagents can be delivered to the reaction regions simultaneously.

Nucleic acid microarrays are preferred. Such microarrays (including nucleic acid chips) are well known in the art (see, for example U.S. Pat. Nos. 5,578,832; 5,861,242; 6,183,698; 6,287,850; 6,291,183; 6,297,018; 6,306,643; and 6,308,170, each incorporated by reference).

Alternatively, antibody microarrays can be produced. The production of such microarrays is essentially as described in Schweitzer & Kingsmore, “Measuring proteins on microarrays”, Curr Opin Biotechnol 2002; 13(1): 14-9; Avseekno et al., “Immobilization of proteins in immunochemical microarrays fabricated by electrospray deposition”, Anal Chem 2001 15; 73(24): 6047-52; Huang, “Detection of multiple proteins in an antibody-based protein microarray system, Immunol Methods 2001 1; 255 (1-2): 1-13.

The present invention also contemplates the preparation of kits for use in accordance with the present invention. Suitable kits include various reagents for use in accordance with the present invention in suitable containers and packaging materials, including tubes, vials, and shrink-wrapped and blow-molded packages.

Materials suitable for inclusion in an exemplary kit in accordance with the present invention comprise one or more of the following: gene specific PCR primer pairs (oligonucleotides) that anneal to DNA or cDNA sequence domains that flank the genetic polymorphisms of interest, reagents capable of amplifying a specific sequence domain in either genomic DNA or cDNA without the requirement of performing PCR; reagents required to discriminate between the various possible alleles in the sequence domains amplified by PCR or non-PCR amplification (e.g., restriction endonucleases, oligonucleotide that anneal preferentially to one allele of the polymorphism, including those modified to contain enzymes or fluorescent chemical groups that amplify the signal from the oligonucleotide and make discrimination of alleles more robust); reagents required to physically separate products derived from the various alleles (e.g. agarose or polyacrylamide and a buffer to be used in electrophoresis, HPLC columns, SSCP gels, formamide gels or a matrix support for MALDI-TOF).

Specifically contemplated are kits comprising two or more polymorphism-specific or allele-specific oligonucleotides or oligonucleotide pairs, wherein each polymorphism-specific or allele-specific oligonucleotide or oligonucleotide pair is directed to one of the polymorphisms recited herein.

For example, the present invention contemplates a kit comprising one or more polymorphism-specific or allele-specific oligonucleotide or oligonucleotide pair directed to one or more of the polymorphisms selected from the group:
rs1489759 A/G in the gene encoding Hedgehog Interacting Protein (HHIP);
rs2240997 G/A in the gene encoding Solute Carrier Family 34 (SLC34A2);
rs7671167 T/C in the Family with sequence similarity 13A (FAM13A) gene;
rs161974 C/T in the gene encoding BICD1;
rs2630578 C/G in the gene encoding BICD1.

It will be appreciated that in this context the term “directed to” means an oligonucleotide or oligonucleotide pair capable of identifying the allele present at the polymorphism.

In one embodiment, the kit comprises one or more polymorphism-specific or allele-specific oligonucleotides or oligonucleotide pairs directed to two or more of the above polymorphisms, while in another embodiment the kit comprises one or more polymorphism-specific or allele-specific oligonucleotides or oligonucleotide pairs directed to all three of the above polymorphisms.

Also, specifically contemplated are kits comprising one or more antibodies to a gene product of one of the polymorphic genes described herein, as are kits comprising one or more microarrays comprising one or more such antibodies, and kits comprising one or more microarrays comprising one or more oligonucleotides described herein.

It will be appreciated that the methods of the invention can be performed in conjunction with an analysis of other risk factors known to be associated with lung cancer. Such risk factors include epidemiological risk factors associated with an increased risk of developing lung cancer. Such risk factors include, but are not limited to smoking and/or exposure to tobacco smoke, age, sex and familial history. These risk factors can be used to augment an analysis of one or more polymorphisms as herein described when assessing a subject's risk of developing lung cancer.

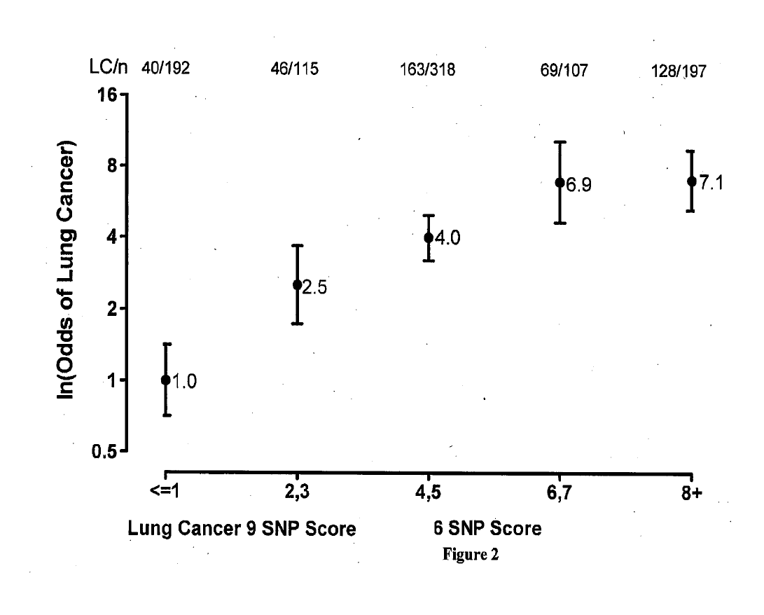

It is recognised that individual SNPs may confer weak risk of susceptibility or protection to a disease or phenotype of interest. These modest effects from individual SNPs are typically measured as odds ratios in the order of 1-3. The specific phenotype of interest may be a disease, such as lung cancer, or an intermediate phenotype based on a pathological, biochemical or physiological abnormality (for example, impaired lung function). As shown herein, when specific genotypes from individual SNPs are assigned a numerical value reflecting their phenotypic effect (for example, a positive value for susceptibility SNPs and a negative value for protective SNPs), the combined effects of these SNPs can be derived from an algorithm that calculates an overall score. Again as shown herein in a case-control study design, this SNP score is linearly related to the frequency of disease (or likelihood of having disease), see for example FIG. 2 herein. The SNP score provides a means of comparing people with different scores and their odds of having disease in a simple dose-response relationship. In this analysis, the people with the lowest SNP score are the referent group (Odds ratio=1) and those with greater SNP scores have a correspondingly greater odds (or likelihood) of having the disease—again in a linear fashion. The Applicants believe, without wishing to be bound by any theory, that the extent to which combining SNPs optimises these analyses is dependent, at least in part, on the strength of the effect of each SNP individually in a univariate analysis (independent effect) and/or multivariate analysis (effect after adjustment for effects of other SNPs or non-genetic factors) and the frequency of the genotype from that SNP (how common the SNP is). However, the effect of combining certain SNPs may also be in part related to the effect that those SNPs have on certain pathophysiological pathways that underlie the phenotype or disease of interest.

The Applicants have found that combining certain SNPs may increase the accuracy of the determination of risk or likelihood of disease in an unpredictable fashion. Specifically, when the distribution of SNP scores for the cases and controls are plotted according to their frequency, the ability to segment those with and without disease (or risk of disease) can be improved according to the specific combination of SNPs that are analysed. It appears that this effect is not solely dependent on the number of relevant SNPs that are analysed in combination, nor the magnitude of their individual effects, nor their frequencies in the cases or controls. It further appears that the ability to improve this segmentation of the population into high and low risk is not due to any specific ratio of susceptibility or protective SNPs. The Applicants believe, without wishing to be bound by any theory, that the greater separation of the population in to high and low risk may at least partly be a function of identifying SNPs that confer a susceptibility or protective phenotype in important but independent pathophysiological pathways.

This observation has clinical utility in helping to define a threshold or cut-off level in the SNP score that will define a subgroup of the population to undergo an intervention. Such an intervention may be a diagnostic intervention, such as imaging test, other screening or diagnostic test (e.g. biochemical or RNA based test), or may be a therapeutic intervention, such as a chemopreventive therapy (for example, cisplatin or etoposide for small cell lung cancer), radiotherapy, or a preventive lifestyle modification (stopping smoking for lung cancer). In defining this clinical threshold, people can be prioritised to a particular intervention in such a way to minimise costs or minimise risks of that intervention (for example, the costs of image-based screening or expensive preventive treatment or risk from drug side-effects or risk from radiation exposure). In determining this threshold, one might aim to maximise the ability of the test to detect the majority of cases (maximise sensitivity) but also to minimise the number of people at low risk that require, or may be are otherwise eligible for, the intervention of interest.

Receiver-operator curve (ROC) analyses analyze the clinical performance of a test by examining the relationship between sensitivity and false positive rate (i.e., 1-specificity) for a single variable in a given population. In an ROC analysis, the test variable may be derived from combining several factors. Either way, this type of analysis does not consider the frequency distribution of the test variable (for example, the SNP score) in the population and therefore the number of people who would need to be screened in order to identify the majority of those at risk but minimise the number who need to be screened or treated. The Applicants have found that this frequency distribution plot may be dependent on the particular combination of SNPs under consideration and it appears it may not be predicted by the effect conferred by each SNP on its own nor from its performance characteristics (sensitivity and specificity) in an ROC analysis.

The data presented herein shows that determining a specific combination of SNPs can enhance the ability to segment or subgroup people into intervention and non-intervention groups in order to better prioritise these interventions. Such an approach is useful in identifying which smokers might be best prioritised for interventions, such as CT screening for lung cancer. Such an approach could also be used for initiating treatments or other screening or diagnostic tests. As will be appreciated, this has important cost implications to offering such interventions.

Accordingly, the present invention also provides a method of assessing a subject's suitability for an intervention diagnostic of or therapeutic for a disease, the method comprising:

a) providing a net score for said subject, wherein the net score is or has been determined by:
i) providing the result of one or more genetic tests of a sample from the subject, and analysing the result for the presence or absence of protective polymorphisms and for the presence or absence of susceptibility polymorphisms, wherein said protective and susceptibility polymorphisms are associated with said disease,
ii) assigning a positive score for each protective polymorphism and a negative score for each susceptibility polymorphism or vice versa;
iii) calculating a net score for said subject by representing the balance between the combined value of the protective polymorphisms and the combined value of the susceptibility polymorphisms present in the subject sample; and

b) providing a distribution of net scores for disease sufferers and non-sufferers wherein the net scores for disease sufferers and non-sufferers are or have been determined in the same manner as the net score determined for said subject;

c) determining whether the net score for said subject lies within a threshold on said distribution separating individuals deemed suitable for said intervention from those for whom said intervention is deemed unsuitable;

wherein a net score within said threshold is indicative of the subject's suitability for the intervention, and wherein a net score outside the threshold is indicative of the subject's unsuitability for the intervention.

The value assigned to each protective polymorphism may be the same or may be different. The value assigned to each susceptibility polymorphism may be the same or may be different, with either each protective polymorphism having a negative value and each susceptibility polymorphism having a positive value, or vice versa.

The intervention may be a diagnostic test for the disease, such as a blood test or a CT scan for lung cancer. Alternatively, the intervention may be a therapy for the disease, such as chemotherapy or radiotherapy, including a preventative therapy for the disease, such as the provision of motivation to the subject to stop smoking.

As described herein, a distribution of SNP scores for lung cancer sufferers and resistant smoker controls (non-sufferers) can be established using the methods of the invention. For example, a distribution of SNP scores derived from the 16 SNP panel consisting of the protective and susceptibility polymorphisms selected from the group consisting of the −133 G/C polymorphism in the Interleukin-18 gene, the −1053 C/T polymorphism in the CYP 2E1 gene, the Arg197gln polymorphism in the Nat2 gene, the −511 G/A polymorphism in the Interleukin 1B gene, the Ala 9 Thr polymorphism in the Anti-chymotrypsin gene, the S allele polymorphism in the Alpha1-antitrypsin gene, the −251 A/T polymorphism in the Interleukin-8 gene, the Lys 751 gln polymorphism in the XPD gene, the +760 G/C polymorphism in the SOD3 gene, the Phe257Ser polymorphism in the REV gene, the Z alelle polymorphism in the Alpha1-antitrypsin gene, the R19W A/G polymorphism in the Cerberus 1 (Cer 1) gene, the Ser307Ser G/T polymorphism in the XRCC4 gene, the K3326 X A/T polymorphism in the BRCA2 gene, the V433M A/G polymorphism in the Integrin alpha-11 gene, and the E375G T/C polymorphism in the CAMKK1 gene, among lung cancer sufferers and non-sufferers is described in PCT/NZ2007/000310 published as WO 2008/048120. As shown therein, a threshold SNP score can be determined that separates people into intervention and non-intervention groups, so as to better prioritise those individuals suitable for such interventions.

The predictive methods of the invention allow a number of therapeutic interventions and/or treatment regimens to be assessed for suitability and implemented for a given subject. The simplest of these can be the provision to the subject of motivation to implement a lifestyle change, for example, where the subject is a current smoker, the methods of the invention can provide motivation to quit smoking.

The manner of therapeutic intervention or treatment will be predicated by the nature of the polymorphism(s) and the biological effect of said polymorphism(s). For example, where a susceptibility polymorphism is associated with a change in the expression of a gene, intervention or treatment is preferably directed to the restoration of normal expression of said gene, by, for example, administration of an agent capable of modulating the expression of said gene. Where a polymorphism is associated with decreased expression of a gene, therapy can involve administration of an agent capable of increasing the expression of said gene, and conversely, where a polymorphism is associated with increased expression of a gene, therapy can involve administration of an agent capable of decreasing the expression of said gene. Methods useful for the modulation of gene expression are well known in the art. For example, in situations where a polymorphism is associated with upregulated expression of a gene, therapy utilising, for example, RNAi or antisense methodologies can be implemented to decrease the abundance of mRNA and so decrease the expression of said gene. Alternatively, therapy can involve methods directed to, for example, modulating the activity of the product of said gene, thereby compensating for the abnormal expression of said gene.

Where a susceptibility polymorphism is associated with decreased gene product function or decreased levels of expression of a gene product, therapeutic intervention or treatment can involve augmenting or replacing of said function, or supplementing the amount of gene product within the subject for example, by administration of said gene product or a functional analogue thereof. For example, where a polymorphism is associated with decreased enzyme function, therapy can involve administration of active enzyme or an enzyme analogue to the subject. Similarly, where a polymorphism is associated with increased gene product function, therapeutic intervention or treatment can involve reduction of said function, for example, by administration of an inhibitor of said gene product or an agent capable of decreasing the level of said gene product in the subject. For example, where a SNP allele or genotype is associated with increased enzyme function, therapy can involve administration of an enzyme inhibitor to the subject.

Likewise, when a protective polymorphism is associated with upregulation of a particular gene or expression of an enzyme or other protein, therapies can be directed to mimic such upregulation or expression in an individual lacking the resistive genotype, and/or delivery of such enzyme or other protein to such individual Further, when a protective polymorphism is associated with downregulation of a particular gene, or with diminished or eliminated expression of an enzyme or other protein, desirable therapies can be directed to mimicking such conditions in an individual that lacks the protective genotype.

The relationship between the various polymorphisms identified above and the susceptibility (or otherwise) of a subject to lung cancer also has application in the design and/or screening of candidate therapeutics. This is particularly the case where the association between a susceptibility or protective polymorphism is manifested by either an upregulation or downregulation of expression of a gene. In such instances, the effect of a candidate therapeutic on such upregulation or downregulation is readily detectable.

For example, in one embodiment existing human lung organ and cell cultures are screened for polymorphisms as set forth above. (For information on human lung organ and cell cultures, see, e.g.: Bohinski et al. (1996) Molecular and Cellular Biology 14:5671-5681; Collettsolberg et al. (1996) Pediatric Research 39:504; Hermanns et al. (2004) Laboratory Investigation 84:736-752; Hume et al. (1996) In Vitro Cellular & Developmental Biology-Animal 32:24-29; Leonardi et al. (1995) 38:352-355; Notingher et al. (2003) Biopolymers (Biospectroscopy) 72:230-240; Ohga et al. (1996) Biochemical and Biophysical Research Communications 228:391-396; each of which is hereby incorporated by reference in its entirety.) Cultures representing susceptibility and protective genotype groups are selected, together with cultures which are putatively “normal” in terms of the expression of a gene which is either upregulated or downregulated where a protective polymorphism is present.

Samples of such cultures are exposed to a library of candidate therapeutic compounds and screened for any or all of: (a) downregulation of susceptibility genes that are normally upregulated in susceptibility polymorphisms; (b) upregulation of susceptibility genes that are normally downregulated in susceptibility polymorphisms; (c) downregulation of protective genes that are normally downregulated or not expressed (or null forms are expressed) in protective polymorphisms; and (d) upregulation of protective genes that are normally upregulated in protective polymorphisms. Compounds are selected for their ability to alter the regulation and/or action of susceptibility genes and/or protective genes in a culture having a susceptibility polymorphisms.

Similarly, where the polymorphism is one which when present results in a physiologically active concentration of an expressed gene product outside of the normal range for a subject (adjusted for age and sex), and where there is an available prophylactic or therapeutic approach to restoring levels of that expressed gene product to within the normal range, individual subjects can be screened to determine the likelihood of their benefiting from that restorative approach. Such screening involves detecting the presence or absence of the polymorphism in the subject by any of the methods described herein, with those subjects in which the polymorphism is present being identified as individuals likely to benefit from treatment.

The methods of the invention are primarily directed at assessing risk of developing lung cancer. Lung cancer can be divided into two main types based on histology—non-small cell (approximately 80% of lung cancer cases) and small-cell (roughly 20% of cases) lung cancer. This histological division also reflects treatment strategies and prognosis.

The non-small cell lung cancers (NSCLC) are generally considered collectively because their prognosis and management is roughly identical. For non-small cell lung cancer, prognosis is poor. The most common types of NSCLC are adenocarcinoma, which accounts for 50% to 60% of NSCLC, squamous cell carcinoma, and large cell carcinoma.

Adenocarcinoma typically originates near the gas-exchanging surface of the lung. Most cases of the adenocarcinoma are associated with smoking. However, adenocarcinoma is the most common form of lung cancer among non-smokers. A subtype of adenocarcinoma, the bronchioalveolar carcinoma, is more common in female non-smokers.

Squamous cell carcinoma, accounting for 20% to 25% of NSCLC, generally originates in the larger breathing tubes. This is a slower growing form of NSCLC.

Large cell carcinoma is a fast-growing form that grows near the surface of the lung. An initial diagnosis of large cell carcinoma is frequently reclassified to squamous cell carcinoma or adenocarcinoma on further investigation.

For small cell lung cancer (SCLC), prognosis is also poor. It tends to start in the larger breathing tubes and grows rapidly becoming quite large. It is initially more sensitive to chemotherapy, but ultimately carries a worse prognosis and is often metastatic at presentation. SCLC is strongly associated with smoking.

Other types of lung cancer include carcinoid lung cancer, adenoid cystic carcinoma, cylindroma, mucoepidermoid carcinoma, and metastatic cancers which originate in other parts of the body and metastasize to the lungs. Generally, these cancers are identified by the site of origin, i.e., a breast cancer metastasis to the lung is still known as breast cancer. Conversely, the adrenal glands, liver, brain, and bone are the most common sites of metastasis from primary lung cancer itself.

Due to the poor prognosis for lung cancer sufferers, early detection is of paramount importance. However, the screening methodologies currently widely available have been reported to be largely ineffective. Regular chest radiography and sputum examination programs were not effective in reducing mortality from lung cancer, leading the authors to conclude that the current evidence did not support screening for lung cancer with chest radiography or sputum cytology, and that frequent chest x-ray screening might be harmful. (See Manser R L, et al., Screening for lung cancer. Cochrane Database of Systematic Reviews 2004, Issue 1. Art. No.: CD001991. DOI: 10.1002/14651858.CD001991.pub2.).

Computed tomography (CT) scans can uncover tumors not yet visible on an X-ray. CT scanning is now being actively evaluated as a screening tool for lung cancer in high risk patients. In a study of over 31,000 high-risk patients, 85% of the 484 detected lung cancers were stage I and were considered highly treatable (see Henschke C I, et al., Survival of patients with stage I lung cancer detected on CT screening. N Engl J. Med., 355(17):1763-71, (2006).

In contrast, a recent study in which 3,200 current or former smokers were screened for 4 years and offered 3 or 4 CT scans reported increased diagnoses of lung cancer and increased surgeries, but no significant differences between observed and expected numbers of advanced cancers or deaths (see Bach P B, et al., Computed Tomography Screening and Lung Cancer Outcomes, JAMA., 297:953-961 (2007)).

It should be noted that screening studies have only been done in high risk populations, such as smokers and workers with occupational exposure to certain substances. A more definitive appraisal of the efficacy of screening using CT may need await the results of ongoing randomized trials in the U.S. and Europe. This is important when one considers that repeated radiation exposure from screening could actually induce carcinogenesis in a small percentage of screened subjects, so this risk should be mitigated by a (relatively) high prevalence of lung cancer in the population being screened. This high prevalence can be achieved by prescreening prior to CT scanning by, for example, the methods described herein.

The invention will now be described in more detail, with reference to the following non-limiting examples.

### Example 1

### Case Association Study

### Introduction

Case-control association studies allow the careful selection of a control group where matching for important risk factors is critical. In this study, smokers diagnosed with lung cancer and smokers without lung cancer with normal lung function were compared. This unique control group is highly relevant as it is impossible to pre-select smokers with zero risk of lung cancer—i.e., those who although smokers will never develop lung cancer. Smokers with a high pack year history and normal lung function were used as a “low risk” group of smokers, as the Applicants believe it is not possible with current knowledge to identify a lower risk group of smokers. The Applicants believe, without wishing to be bound by any theory, that this approach allows for a more rigorous comparison of low penetrant, high frequency polymorphisms that may confer an increased risk of developing lung cancer. The Applicants also believe, again without wishing to be bound by any theory, that there may be polymorphisms that confer a degree of protection from lung cancer which may only be evident if a smoking cohort with normal lung function is utilised as a comparator group. Thus smokers with lung cancer would be expected to have a lower frequency of these polymorphisms compared to smokers with normal lung function and no diagnosed lung cancer.

### Methods

### Subject Recruitment

Subjects of European descent who had smoked a minimum of fifteen pack years and diagnosed with lung cancer were recruited. Subjects met the following criteria: diagnosed with lung cancer based on radiological and histological grounds, including primary lung cancers with histological types of small cell lung cancer, squamous cell lung cancer, adenocarcinoma of the lung, non-small cell cancer (where histological markers can not distinguish the subtype) and broncho-alveolar carcinoma. Subjects could be of any age and at any stage of treatment after the diagnosis had been confirmed. 454 subjects were recruited, of these 53% were male, the mean FEV1/FVC (1SD) was 64% (13), mean FEV1 as a percentage of predicted was 73 (22). Mean age, cigarettes per day and pack year history was 69 yrs (10), 20 cigarettes/day (10) and 41 pack years (25), respectively.

Lung cancer cohort: Subjects with lung cancer were recruited from a tertiary hospital clinic, aged >40 yrs and the diagnosis confirmed through histological or cytological specimens in 95% of cases. Non-smokers with lung cancer were excluded from the study and only primary lung cancer cases with the following pathological diagnosis were included: adenocarcinoma, squamous cell cancer, small cell cancer and non-small cell cancer (generally large cell or bronchioalveolar subtypes). Lung function measurement (pre-bronchodilator) was performed within 3 months of lung cancer diagnosis, prior to surgery and in the absence of pleural effusions or lung collapse on plain chest radiographs. For lung cancer cases that had already undergone surgery, pre-operative lung function performed by the hospital lung function laboratory was sourced from medical records.

COPD cohort: Subjects with COPD were identified through hospital specialist clinics as previously described. Subjects recruited into the study were aged 40-80 yrs, with a minimum smoking history of 20 pack-yrs and COPD confirmed by a respiratory specialist based on pre-bronchodilator spirometric criteria (Gold stage 2 or more).

Control cohort: Control subjects were recruited based on the following criteria: aged 45-80 yrs and with a minimum smoking history of 20 pack-yrs. Control subjects were volunteers who were recruited from the same patient catchment area (suburb) as those serving the lung cancer and COPD hospital clinics through either (a) a community postal advert or (b) while attending community-based retired military/servicemen's clubs. Controls with COPD, based on spirometry (GOLD stage 1 or more), who constituted 35% of the smoking volunteers, were excluded from further analysis.

488 European subjects who had smoked a minimum of twenty pack years and who had never suffered breathlessness and had not been diagnosed with an obstructive lung disease or lung cancer in the past were also studied. This control group was recruited through clubs for the elderly and consisted of 60% male, the mean FEV1/FVC (1SD) was 78% (7), mean FEV1 as a percentage of predicted was 99. Mean age, cigarettes per day and pack year history was 65 yrs (10), 24 cigarettes/day (11) and 40 pack years (19), respectively.

All participants gave written informed consent, and underwent blood sampling for DNA extraction, spirometry and an investigator-administered questionnaire. Spirometry was performed using a portable spirometer (Easy-One™; ndd Medizintechnik AG, Zurich, Switzerland). Lung function conformed to American Thoracic Society (ATS) standards for reproducibility, with the highest value of the best three acceptable blows used for classification of COPD status. COPD was defined according to Global Initiative for Chronic Obstructive Lung Diseases (GOLD) 2 or more criteria (FEV1/FVC<70% and FEV1% predicted ≦80%) using pre-bronchodilator spirometric measurements [www.goldcopd.com]. A modified ATS respiratory questionnaire was administered to all cases and controls, which collected data on demographic variables such as age, sex, medical history, family history of lung disease, active and passive tobacco exposure, respiratory symptoms and occupational aero-pollutant exposures. The study was approved by the Multi Centre Ethics Committee (New Zealand).

Using a PCR based method (Sandford et al., 1999), all subjects were genotyped for the α1-antitrypsin mutations (S and Z alleles) and those with the ZZ allele were excluded. On regression analysis, the age difference and pack years difference observed between lung cancer sufferers and resistant smokers was found not to determine FEV or lung cancer.

This study shows that polymorphisms found in greater frequency in lung cancer patients compared to resistant smokers may reflect an increased susceptibility to the development of lung cancer. Similarly, polymorphisms found in greater frequency in resistant smokers compared to lung cancer may reflect a protective role.

Summary of characteristics for the lung cancer sufferors, COPD
controls and resistant smoking controls.

| | | | | | Control | |
|---|---|---|---|---|---|---|
| Parameter | Lung Cancer | | COPD | | smokers | |
| Mean (1 SD) | N = 454 | | N = 458 | | N = 488 | |
| % male | 53% | | 59% | | 60% | |
| Age (yrs) | 69 | (10) | 66 | (9) | 65 | (10) |

Smoking history

| Current smoking (%) | 35% | | 40% | | 48% | |
|---|---|---|---|---|---|---|
| Age started (yr) | 18 | (4) | 17 | (3) | 17 | (3) |
| Yrs smoked | 41 | (12) | 42 | (11) | 35 | (11) |
| Pack years* | 41 | (25) | 47 | (20) | 40 | (19) |
| Cigarettes/day | 20 | (10) | 23 | (9) | 24 | (11) |
| Yrs since quitting | 11.4 | (6.7) | 9.8 | (7.4) | 13.9 | (8.1) |

History of other exposures

| | | | |
|---|---|---|---|
| Work dust exposure* | 63% | 59% | 47% |
| Work fume exposure | 41% | 40% | 38% |
| Asbestos exposure* | 23% | 22% | 16% |

Family history

| | | | |
|---|---|---|---|
| FHx of COPD | 33% | 37% | 28% |
| FHx of lung cancer* | 19% | 11% | 9% |

Lung function

| | | | | | | |
|---|---|---|---|---|---|---|
| FEV1 (L)* | 1.86 | (0.48) | 1.25 | (0.48) | 2.86 | (0.68) |

| FEV1 % predicted* | 73% | | 46% | 99% | |
|---|---|---|---|---|---|
| FEV1/FVC* | 64 | (13) | 46% | 78 | (7) |
| | | | (8) | | |

| | | | |
|---|---|---|---|
| Spirometric COPD#* | 57% | 100% | 0% |

ETS = environmental tobacco smoke,
#According to GOLD 2+ criteria,
*P < 0.05.

### Genotyping Methods

Genomic DNA was extracted from whole blood samples using standard salt-based methods and purified genomic DNA was aliquoted (10 ng·μL⁻¹ concentration) into 96-well or 384-well plates. Samples were genotyped using either the Sequenom™ system (Sequenom™ Autoflex Mass Spectrometer and Samsung 24 pin nanodispenser) or Taqman® SNP genotyping assays (Applied Biosystems, USA) utilising minor groove-binder probes. Taqman® SNP genotyping assays were run in 384-well plates according to the manufacturer's instructions. PCR cycling was performed on both GeneAmp® PCR System 9700 and 7900HT Fast Real-Time PCR System (Applied Biosystems, USA) devices.

The SNPs typed using the Applied Biosystems 7900HT Fast Real-Time PCR System used genomic DNA extracted from white blood cells and diluted to a concentration of 10 ng/μL, containing no PCR inhibitors, and having an A260/280 ratio greater than 1.7. The reaction mix for each assay was first prepared according to the following table. Enough reaction mix was made to account for all No Template Controls (NTCs) and samples with a surplus 10% to account for pipetting losses. All solutions were kept on ice for the duration of the experiment.

Reaction Mix

| | Volume (μl) | |
|---|---|---|
| Reagent | One Reaction | n Reactions |
| TaqMan Genotyping Master Mix (2×) | 2.50 | n × 2.50 + 10% |
| SNP Genotyping Assay Mix (40×) | 0.125 | n × 0.125 + 10% |
| DNase-free water | 1.375 | n × 1.375 + 10% |
| Total Volume | 4.00 | |

The reaction plate was then prepared. First, 1 μL of the NTC (DNase-free water) and DNA samples were pipetted into the appropriate wells of the 384-well reaction plate. Each reaction mix was inverted and spun down to mix, and then 4 μL of the reaction mix was added to the appropriate wells of the reaction plate. The reaction plate was then covered with an optical adhesive cover and then briefly centrifuged to spin down contents and eliminate air bubbles. Once preparation of the reaction plate was complete the plate was kept on ice and covered with aluminium foil to protect from the light until it is loaded into the 7900HT Real-Time PCR System.

Sequences were designed according to the following sequences.

rs16969968 (nACHRa3/5)

[Seq ID NO. 1]

TAGAAACACATTGGAAGCTGCGCTC[A/G]ATTCTATTCGCTACATTACA
AGACA
rs2202507 (GYPA)

[Seq ID NO. 2]

AGACGACACTAGTTTTTAAAGTTTT[G/T]ATTAATCGCTGCTGTGAAGC
TGCAT
rs1489759 (HHIP)

[Seq ID NO. 3]

GAAATTGTTTTCTTTGGACAACTTG[A/G]CAAAAACCAATCATCTGTCA
GTGAT
rs2808630 (CRP)

[Seq ID NO. 4]

AGGCCAGAGGCTGTCTACCAGACTA[C/T]GTATAGTAAGATGCAAGCAA
CTGAA
rs2240997 (SLC34A2)

[Seq ID NO. 5]

CAGGAGTTCATATCTAGAGAGCTGT[A/G]AGTCAGGCCTTCCTTCTTAG
CGGGT
rs 1051730 (nAChRa3/5)

[Seq ID NO. 6]

AGCAGTTGTACTTGATGTCGTGTTT[A/G]TAGCCTGGGGCTTTGATGAT
GGCCC
rs 1052486 (BAT3)

[Seq ID NO. 7]

GTGATGGTGGGAGAAGCCACACCAG[A/G]CCCTCCAGCCCCTGGCCCTG
CAGGC
rs1422795 (ADAM19)

[Seq ID No. 8]

TGGGCAAGCAGCTTGCGCCTCCAAC[C/T]GAGAAAGGACCAGAGGGTAG
AATAT
rs7671167 (FAM13A)

[Seq ID No. 9]

CATTAAGAAAGAATTAGGTAAATTC[C/T]AAAACATAAGGGAATACTAT
GACAA

After the plate was pre-read with the allelic discrimination document, the amplification run was completed (whether using the 7900HT Real-Time PCR System or another thermal cycler), and after the allelic discrimination post-read was completed the plate was analysed. Automatic calls made by the allelic discrimination document were reviewed using the AQ curve data. The allele calls made on the genotypes were then converted into genotypes.

The Family with sequence similarity 13A (FAM13A) SNP (rs7671167) on 4q22, the hedgehog-interacting protein (HHIP) SNP (rs1489759) on 4q31, the glycophorin A (GYPA) SNP (rs2202507) on 4q31, the C-reactive protein (CRP) SNP (rs2808630) on 1q21, the glutathione S-transferase C-terminal domain (GSTCD) SNP (rs 2808630) on 4q42, the A Disintegrin and Metalloproteinase 19 (ADAM19) SNP (rs 1422795) on 5q33, the receptor for advanced glycation end-products (AGER) SNP (rs 2070600) on 6p21 and the G-protein receptor 126 (GPR126) SNP (rs 11155242) on 6q24 were also genotyped by Taqman® SNP genotyping assays.

The nicotinic acetylcholine receptor (nAChR) SNP (rs16969968) on 15q25, the HLA-B associated transcript (BAT3) SNP (rs 1052486) on 6p21 and the cisplatin-resistance regulated gene 9 (CRR9/TERT) rs 402710 SNP (on 5p15 and in LD with rs401681) were also genotyped using the Sequenom™ system.

Failed samples were repeated until call rates of ≧95% for each SNP in each cohort were achieved. Genotype frequencies for each SNP were compared between the 3 primary groups (control smokers, COPD and lung cancer cohorts) and with sub-phenotyping the lung cancer cohort according to the presence or absence of COPD (based on both GOLD 1 and GOLD 2 criteria).

Algorithm and Susceptibility Score

The cumulative effect of those SNP genotypes identified as susceptible (Odds ratio, OR>1) or protective (OR<1), based on significant distortions in frequency (P<0.05) between the cases and the control smokers, was examined Only the lung cancer and control smoker cohorts were used for this analysis. In this algorithm, for each subject, a numerical value of −1 was assigned for each of the protective genotypes present among the protective SNPs and +1 for each of the susceptible genotypes present. Where an individual did not have either the protective or susceptibility genotype for that SNP, the score was 0 (i.e. did not contribute to the genetic score). Weighting the presence of specific susceptible or protective genotypes according to their individual odds ratios (ORs; from univariate regression) did not significantly improve the discriminatory performance of the cumulative SNP score (unpublished data).

The algorithmic approach used here involved deriving an overall “susceptibility score” for each subject (from the control and lung cancer cohorts) by combining genetic data (cumulative SNP scores) and the clinical variables age >60 years of age (score, +4), family history of lung cancer (score, +3) and prior diagnosis of COPD (score, +4). By using multivariate logistic and stepwise regression analysis, the 9-SNP panel was examined in combination with the pre-stipulated clinical variables above. As smoking exposure (pack-years) was a recruitment criterion for this study, and comparable between cases and controls, it was not included in the scoring system described here. The lung cancer susceptibility score (for the control and lung cancer cohorts) was plotted with (a) the frequency of lung cancer and (b) the floating absolute risk (equivalent to OR) across the combined smoker/ex-smoker cohort.

Analysis

Patient characteristics in the cases and controls were compared by ANOVA for continuous variables and Chi-squared test for discrete variables (Mantel-Haenszel, odds ratio (OR)). Genotype and allele frequencies were checked for each SNP by Hardy-Weinberg Equilibrium (HWE). Population admixture across cohorts was performed using structure analysis on genotyping data from 40 unrelated SNPs. Distortions in the genotype frequencies were identified by comparing lung cancer (sub-phenotyped by COPD) and/or COPD cases with “resistant” smoking controls using two-by-two contingency tables.

Genotype data (9-SNP panel) and the clinical variables were combined in a stepwise logistic regression to assess their relative effects on discriminating low and high risk (by point estimate and receiver operating characteristic (ROC) curve) by score quintile. The frequency distribution of the lung cancer susceptibility score was compared across the cases and controls. Its clinical utility was assessed using ROC analysis, which assesses how well the model predicts risk across the score (i.e. clinical performance of the score with respect to sensitivity, specificity and false positive rate).

### Results

The following tables show the results of univariate analysis of the polymorphisms described herein.

TABLE 1
Nicotinic Acetylcholine receptor subunit alpha 3/5 (nAChR)
rs16969968 G/A polymorphism allele and genotype frequencies in
control smokers and those with lung cancer

| | Allele | | Genotype | | |
|---|---|---|---|---|---|
| Cohort | G | A | GG | GA | AA |
| Control smokers | 655 | 295 | 225 | 205 | 45 |
| N = 475 | (69%) | (31%) | (47%) | (43%) | (9%) |
| Lung Cancer | 539 | 335 | 170 | 199 | 68 |
| N = 437 | (62%) | (38%) | (39%) | (46%) | (16%) |

Genotype: The AA genotype of the nAChR rs16969968 G/A polymorphism was present at greater frequency in those with lung cancer compared to control smokers, 16% vs 9%, respectively (OR = 1.8 (95% confidence interval 1.2-2.7), λ² = 7.8, P = 0.005).
AA = susceptible genotype for lung cancer.
Allele: The A allele of the nAChR rs16969968 G/A polymorphism was present at greater frequency in those with lung cancer compared to control smokers, 38% vs 31%, respectively (OR = 1.4 (95% confidence interval 1.1-1.7), λ² = 10.7, P = 0.001).
A = susceptible allele for lung cancer.

TABLE 2
nAChR rs1051730 C/T polymorphism allele and genotype frequencies
in control smokers and those with lung cancer

| | Allele | | Genotype | | |
|---|---|---|---|---|---|
| Cohort | C | T | CC | CT | TT |
| Control smokers | 659 | 293 | 227 | 205 | 44 |
| N = 476 | (69%) | (31%) | (48%) | (43%) | (9%) |
| Lung cancer | 540 | 338 | 171 | 198 | 70 |
| N = 439 | (62%) | (38%) | (39%) | (45%) | (16%) |

Genotype: The TT genotype of the nAChR rs1051730 C/T polymorphism was present at greater frequency in those with lung cancer compared to control smokers, 16% vs 9%, respectively (OR = 1.9 (95% confidence interval 1.2-2.8), λ² = 9.4, P = 0.002).
TT = susceptible genotype for lung cancer.
Allele: The T allele of the nAChR rs1051730 C/T polymorphism was present at greater frequency in those with lung cancer compared to control smokers, 38% vs 31%, respectively (OR = 1.4 (95% confidence interval 1.2-1.7), λ² = 12.0, P = 0.0005).
T = susceptible allele for lung cancer.

### Note:

The rs16969968 SNP is reported to be in linkage disequilibrium with the rs1051730 polymorphism, and these two SNPs are estimated to be about 11 kb apart.

When the rs16969968 genotype (GG, GA, or AA) for each subject in the combined cohort of controls and lung cancer patients (n=912) was compared with their rs1051730 SNP genotype (CC, CT, or TT), a nearly complete concordance of 99.8% (910/912) was observed. This means that in a risk assessment for lung cancer, either SNP could be used in a panel of SNPs because they are effectively interchangeable and confer the same level of risk (as shown in the univariate analyses above). The small observed variation is due to slightly different numbers in each group.

TABLE 3
Hedgehog Interacting Protein (HHIP) rs1489759 A/G polymorphism
allele and genotype frequencies in control smokers and those
with lung cancer

| | Allele | | Genotype | | |
|---|---|---|---|---|---|
| Cohort | A | G | AA | AG | GG |
| Control smokers | 579 | 389 | 178 | 223 | 83 |
| N = 484 | (60%) | (40%) | (37%) | (46%) | (17%) |
| Lung Cancer | 563 | 327 | 174 | 215 | 56 |
| N = 445 | (63%) | (37%) | (39%) | (48%) | (13%) |

Genotype: The GG genotype of the HHIP rs1489759 A/G polymorphism was present at reduced frequency in those with lung cancer compared to control smokers, 13% vs 17%, respectively (OR = 0.70 (95% confidence interval 0.47-1.0), λ² = 3.79, P = 0.05).
GG = protective genotype for lung cancer.

TABLE 4
Glycophorin A Precursor Gene (GYPA) rs2202507 A/C polymorphism
allele and genotype frequencies in control smokers and those
with lung cancer

| | Allele | | Genotype | | |
|---|---|---|---|---|---|
| Cohort | A | C | AA | AC | CC |
| Control smokers | 489 | 471 | 138 | 213 | 129 |
| N = 480 | (51%) | (49%) | (29%) | (44%) | (27%) |
| Lung Cancer | 465 | 413 | 116 | 233 | 90 |
| N = 439 | (53%) | (47%) | (26%) | (53%) | (21%) |

Genotype: The CC genotype at the GYPA rs2202507 A/C polymorphism was present at reduced frequency in those with lung cancer compared to control smokers, 21% vs 27%, respectively (OR = 0.70 (95% confidence interval 0.51-0.97), λ² = 5.13, P = 0.02).
CC = protective genotype for lung cancer.

TABLE 5
Solute Carrier Family 34 (SLC34A2) rs2240997 G/A polymorphism
allele and genotype frequencies in control smokers and those with
lung cancer

| | Allele | | Genotype | | |
|---|---|---|---|---|---|
| Cohort | G | A | GG | GA | AA |
| Control smokers | 872 | 88 | 397 | 78 | 5 |
| N = 480 | (91%) | (9%) | (83%) | (16%) | (1%) |
| Lung Cancer | 770 | 112 | 334 | 102 | 5 |
| N = 441 | (87%) | (13%) | (76%) | (23%) | (1%) |

Genotype: The GA/AA genotype at the Solute Carrier Family 34 (SLC34A2) rs2240997 polymorphism was present at greater frequency in those with lung cancer compared to control smokers, 24% vs 17%, respectively (OR = 1.53 (95% confidence interval 1.1-2.1), λ² = 6.81, P = 0.009).
GA/AA = susceptible genotype for lung cancer.
Allele: The A allele of the Solute Carrier Family 34 (SLC34A2) rs 2240997 polymorphism was present at greater frequency in those with lung cancer compared to control smokers, 13% vs 9%, respectively (OR = 1.4 (95% confidence interval 1.1-2.0), λ² = 5.92, P = 0.01).
A = susceptible allele for lung cancer.

TABLE 6
HLA-B associated transcript 3 (BAT3) rs1052486 A/G polymorphism
allele and genotype frequencies in control smokers and those
with lung cancer

| | Allele | | Genotype | | |
|---|---|---|---|---|---|
| Cohort | A | G | AA | AG | GG |
| Control smokers | 477 | 455 | 119 | 239 | 108 |
| N = 466 | (51%) | (49%) | (26%) | (51%) | (23%) |
| Smokers with COPD | 476 | 408 | 127 | 222 | 93 |
| N = 442* | (54%) | (46%) | (29%) | (50%) | (21%) |
| Lung Cancer | 434 | 442 | 112 | 210 | 116 |
| N = 438 | (50%) | (50%) | (26%) | (48%) | (27% |

*Smokers with COPD provide an additional control group for those at risk of lung cancer from smoking.
Genotype: The GG genotype at the HLA-B associated transcript 3 (BAT3) rs1052486 polymorphism was present at greater frequency in those with lung cancer compared to smokers with COPD controls, 27% vs 21%, (OR = 1.4 (95% confidence interval 0.98-1.87), λ² = 3.6, P = 0.08 compared to AA/GG).
GG = susceptible genotype for lung cancer.
Allele: the G allele of the HLA-B associated transcript 3 (BAT3) rs1052486 polymorphism was present at greater frequency in those with lung cancer compared to COPD controls, 50% vs 46%, respectively (OR = 1.2 (95% confidence interval 0.98-1.4), λ² = 3.26, P = 0.07).
G = susceptible allele for lung cancer.

TABLE 7
C reactive protein (CRP) T/C rs2808630 polymorphism allele and
genotype frequencies in control smokers and those with lung cancer

| | Allele | | Genotype | | |
|---|---|---|---|---|---|
| Cohort | T | C | TT | TC | CC |
| Control smokers | 655 | 311 | 225 | 205 | 53 |
| N = 483 | (68%) | (32%) | (47%) | (42%) | (11%) |
| Lung Cancer | 621 | 261 | 214 | 193 | 34 |
| N = 441 | (70%) | (30%) | (49%) | (44%) | (8%) |

Genotype: The CC genotype at the C reactive protein (CRP) T/C rs2808630 polymorphism was present at reduced frequency in those lung cancer cases compared to controls, 8% vs 11%, (OR = 0.68 (95% confidence interval 0.42-1.1), λ² = 2.9, P = 0.09 compared to TT/TC).
CC = protective genotype for lung cancer.

TABLE 7a
CRP rs2808630 Lung Cancer Subgroup Analyses

| Cohort | TT | TC | CC | OR* (95%) | P |
|---|---|---|---|---|---|
| LC + COPD, | 99 (48%) | 90 (43%) | 18 (9%) | 0.77 (0.42-1.40) | 0.37 |
| N = 207 | | | | | |
| LC only, | 106 (52%) | 85 (42%) | 11 (5%) | 0.47 (0.22-0.95) | 0.02 |
| N = 202 | | | | | |

*CC vs TC/TT compared to matched smoking controls (Mantel-Haenszel)
After stratification of the lung cancer cohort by available spirometric data (n = 409) into those with and without COPD (according to GOLD ≧2 criteria) a significant association of the CC genotype with the lung cancer only group was identified (11% in controls vs 5%, OR 0.47, P = 0.02). The frequency of the CC genotype was significantly lower in the lung cancer only cohort compared to lung cancer with COPD (5% vs 9%, OR = 0.54, P = 0.03).
CC = protective genotype for lung cancer in absence of COPD.

TABLE 8
Genotype frequencies for the CRR9 rs401681 polymorphism
in the lung cancer cohort compared to smoking controls.

| Primay Cohorts | | | | OR* | P |
|---|---|---|---|---|---|
| (call rate %) | AA | AG | GG | (95% CI) | value* |
| Control smokers | 41 (8%) | 230 (47%) | 216 (44%) | — | — |
| N = 487 (99%) | | | | | |
| Lung cancer | 43 (9%) | 198 (44%) | 212 (47%) | 1.10 | 0.45 |
| N = 453 (99%) | | | | (0.85-1.44) | |

Lung Cancer Subgroup Analyses

| | | | | | |
|---|---|---|---|---|---|
| LC + COPD, | 19 (8%) | 106 (49%) | 90 (42%) | 0.90 | 0.54 |
| N = 215 | | | | (0.64-1.27) | |
| LC only, | 21 (10%) | 77 (37%) | 109 (53%) | 1.4 | 0.05 |
| N = 207 | | | | (0.99-1.96) | |

*GG vs AG/AA compared to matched smoking controls (Mantel-Haenszel)
When the lung cancer cases were divided according to their spirometry (n = 422) into those with COPD and without COPD (i.e., sub-grouped by pre-operative lung function) according to GOLD ≧2 criteria, the frequency of the GG genotype of the CRR9 rs401681 polymorphism was 42% in lung cancer with COPD (vs 44% in controls, OR = 0.90, P = 0.54) and 53% in lung cancer only subjects (vs 44% in controls, OR = 1.40, P = 0.05) respectively (Table 8). The GG genotype was raised in the lung cancer only patients compared to the lung cancer with COPD group (53% vs 42%, OR = 1.54, P = 0.03). The GG genotype of the TERT/CRR9 rs401681 polymorphism confers susceptibility to lung cancer in the absence of COPD.
GG = susceptible genotype for lung cancer in absence of COPD.

CRR9 rs402710
The CRR9 rs402710 polymorphism is reported to be in 100% LD with the rs401681 polymorphism. The CRR9 rs402710 polymorphism was also analysed, and yielded identical allele and genotype frequencies across the various cohorts to those shown in Table 8 above. That is, the GG genotype frequency of the TERT/CRR9 (rs402710) SNP was 47% in lung cancer cases compared to controls (44%, OR=1.10, P=0.45). When the lung cancer cases were divided according to their spirometry (n=422), the frequency of the GG genotype was 42% in lung cancer with COPD (vs. 44% in controls, OR=0.90, P=0.54) and 53% in lung cancer only subjects (vs. 44% in controls, OR=1.40, P=0.05) respectively (Table 8). The GG genotype was raised in the lung cancer only patients compared to the lung cancer with COPD group (53% vs. 42%, OR=1.54, P=0.03). The GG genotype of the TERT/CRR9 rs402710 polymorphism confers susceptibility to lung cancer in the absence of COPD.

GG=susceptible genotype for lung cancer in absence of COPD.

This demonstrates that a polymorphism in LD with one of the recited polymorphism may be substituted for that polymorphism and remain informative in the methods of the present invention.

TABLE 9
Genotype frequencies for the ADAM19 rs1422795 polymorphism in the lung
cancer cohort compared to smoking controls.

| Primary Cohorts (call rate %) | TT | TC | CC | OR* (95% CI) | P value* |
|---|---|---|---|---|---|
| Controls N = 486 (99%) | 213 (44%) | 227 (47%) | 46 (9%) | — | — |
| Lung cancer N = 451 (98%) | 183 (41%) | 210 (47%) | 58 (13%) | 1.41 (0.92-2.17) | 0.10 |

*CC vs TC/CC compared to matched smoking controls (Mantel-Haenszel)
Genotype: The CC genotype at the ADAM19 rs1422795 polymorphism was present at increased frequency in those with lung cancer compared to controls, 13% vs 9%, (OR = 1.41 (95% confidence interval 0.92-2.17), P = 0.10 compared to TT/TC).
CC = susceptible genotype for lung cancer

TABLE 10
Genotype frequencies for the FAM13A1 rs 7671167 SNP in the lung cancer
cohort compared to smoking controls.

| Primary Cohorts (call rate %) | TT | TC | CC | OR* (95% CI) | P value* |
|---|---|---|---|---|---|
| Controls N = 485 (99%) | 100 (21%) | 240 (49%) | 145 (30%) | — | — |
| Lung cancer N = 449 (99%) | 118 (26%) | 235 (52%) | 96 (21%) | 0.64 (0.47-0.87) | 0.003 |

*CC vs TC/CC compared to matched smoking controls (Mantel-Haenszel)
Genotype: The CC genotype at the FAM13A1 rs7671167 polymorphism was present at reduced frequency in those lung cancer cases compared to controls, 21% vs 30%, (OR = 0.64 (95% confidence interval 0.47-0.87), P = 0.003 compared to TT/TC).
CC = protective genotype for lung cancer.

TABLE 11
BICD1 rs161974 C/T polymorphism allele and genotype frequencies
in control smokers and those with lung cancer

| Primary | | | Odds | | | | Odds |
|---|---|---|---|---|---|---|---|
| Cohorts | | | Ratio^{#} | | | | Ratio* |
| (call | | | (95% CI)^{#} | | | | (95% CI)* |
| rate %) | G | C | P value^{#} | GG | CG | CC | P value* |
| Controls | 799 | 171 | — | 330 | 139 | 16 | — |
| N = 485 | (82%) | (18%) | | (68%) | (29%) | (3%) | |
| (99%) | | | | | | | |
| Lung | 736 | 162 | 1.03 | 313 | 110 | 26 | 1.80 |
| cancer | (82%) | (18%) | (0.81-1.31) | (70%) | (24%) | (6%) | (0.92-3.57) |
| N = 449 | | | 0.82 | | | | 0.067 |
| (100%) | | | | | | | |

*CC vs CG/GG compared to matched smoking controls (Mantel-Haenszel)
Allele: The C allele at the BICD1 rs161974 C/T polymorphism was present at increased frequency in lung cancer cases compared to controls, 63% vs 58%, (OR = 1.24 (95% confidence interval 1.03-1.50), P = 0.022 compared to T).
C = susceptibility allele for lung cancer.
T = protective allele for lung cancer.

TABLE 12
BICD1 rs2630578 C/G polymorphism allele and genotype frequencies in control
smokers and those with lung cancer

| | | | | | | | Odds | Odds |
|---|---|---|---|---|---|---|---|---|
| | | | Odds Ratio# | | | | Ratio* | Ratio |
| Primary Cohorts | | | (95% CI)^{#} | | | | (95% CI)* | (95% CI) |
| (call rate %) | G | C | P value^{#} | GG | CG | CC | P value* | P value^{$} |
| Controls | 799 | 171 | — | 330 | 139 | 16 | — | — |
| N = 485 (99%) | (82%) | (18%) | | (68%) | (29%) | (3%) | | |
| COPD | 778 | 138 | 0.83 | 329 | 120 | 9 | — | — |
| N = 458 (99%) | (85%) | (15%) | (0.64-1.07) | (72%) | (26%) | (2%) | | |
| | | | 0.13 | | | | | |
| Lung cancer | 736 | 162 | 1.03 | 313 | 110 | 26 | 1.80 | 3.07¹ |
| N = 449 (100%) | (82%) | (18%) | (0.81-1.31) | (70%) | (24%) | (6%) | (0.92-3.57) | (1.35-7.13) |
| | | | 0.82 | | | | 0.067 | 0.003 |
| Controls + COPD | 1577 | 309 | — | 659 | 259 | 25 | — | — |
| N = 943 | (84%) | (16%) | | (70%) | (27%) | (3%) | | |
| Lung cancer | 736 | 162 | 1.12 | 313 | 110 | 26 | 2.26² | |
| N = 449 | (82%) | (18%) | (0.91-1.39) | (70%) | (24%) | (6%) | (1.24-4.10) | |
| (100%) | | | 0.28 | | | | 0.004 | |

*CC vs CG/GG - lung cancer compared to matched smoking controls (Mantel-Haenszel)
¹CC vs CG/GG - lung cancer compared to COPD (Mantel-Haenszel),),
²CC vs CG/GG - lung cancer compared to COPD + controls (Mantel-Haenszel)
^{#}C vs G compared to matched smoking controls (Mantel-Haenszel)
Genotype: The CC genotype at the BICD1 rs2630578 C/G polymorphism was present at increased frequency in lung cancer cases compared to controls, 6% vs 3%, (OR = 1.80 (95% confidence interval 0.9-3.57), P = 0.067 compared to CG/GG).
CC = susceptibility genotype for lung cancer.

Comparison of the lung cancer cohort against all matched smoking controls (resistant smokers+COPD sufferers) confirmed a significant association of the CC genotype with susceptibility to lung cancer, where the frequency of the CC genotype was significantly greater in the lung cancer cohort compared to smoking controls (3% in controls vs. 6%, OR 2.26, P=0.004).

### Example 2

### 4 SNP Panel

Genotype type data for many SNPs can be combined according to an algorithm where the presence of a susceptibility genotype is assigned a positive score, while the presence of a protective genotype is assigned a negative score. This allows genotype data for a panel of SNPs to be combined to generate a score indicating a level of susceptibility to lung cancer. This score is referred to herein as the lung cancer susceptibility (LCS) score.

This example presents an analysis of distributions of LCS scores derived for lung cancer sufferers and control resistant smokers using a 4 SNP panel as described below.

LCS scores for each subject were derived by assigning a score of +1 for the presence of each susceptibility genotype, or −1 for the presence of each protective genotype in the 4 SNP panel. The 4 SNP panel comprised the nAChR rs16969968 G/A polymorphism, the HHIP rs1489759 A/G polymorphism, and the GYPA rs2202507 A/C polymorphism, the Solute Carrier Family 34 (SLC34A2) rs 2240997 polymorphisms. The scores were added to derive the 4 SNP panel LCS score for each subject. Table 13 below shows the distribution of LCS scores derived from the 4 SNP panel amongst the lung cancer patients and the resistant smoker controls.

TABLE 13
Lung cancer susceptibility score from the 4 SNP panel

| | Low risk score | | Neutral | High risk scores |
|---|---|---|---|---|
| Score | −2 | −1 | 0 | 1 or 2 |
| Controls | 51 | 71 | 285 | 79 |
| N = 286 | (10%) | (15%) | (59%) | (17%) |
| Lung cancer | 24 | 56 | 249 | 120 |
| N = 449 | (5%) | (13%) | (56%) | (27%) |

The frequency of high risk scores and low risk scores in lung cancer patients compared to controls was 27% vs. 17% (high risk), and 18% vs. 25% (low risk), respectively (OR=2.32 (95% confidence interval of 1.5-3.5λ²=17.1, P<0.0001).

### Example 3

### 5 SNP Panel

This example presents an analysis of distributions of LCS scores derived for lung cancer sufferers and control resistant smokers using a 5 SNP panel as described below.

LCS scores for each subject were derived by assigning a score of +1 for the presence of each susceptibility genotype, or −1 for the presence of each protective genotype in the 5 SNP panel. The 5 SNP panel comprised the nAChR rs16969968 G/A polymorphism, the HHIP rs1489759 A/G polymorphism, the GYPA rs2202507 A/C polymorphism, the Solute Carrier Family 34 (SLC34A2) rs 2240997, and the HLA-B associated transcript 3 (BAT3) rs 1052486 A/G polymorphisms. The scores were added to derive the 5 SNP panel LCS score for each subject. Table 14 below shows the distribution of LCS scores derived from the 5 SNP panel amongst the lung cancer patients and the resistant smoker controls.

TABLE 14
Lung cancer susceptibility score from the 5 SNP panel

| | Low risk score | | Neutral | High risk scores |
|---|---|---|---|---|
| Score | −2 | −1 | 0 | 1 or 2 or 3 |
| Controls | 39 | 69 | 240 | 138 |
| N = 486 | (8%) | (14%) | (49%) | (28%) |
| Lung cancer | 16 | 56 | 199 | 178 |
| N = 449 | (4%) | (12%) | (44%) | (40%) |

The frequency of high risk scores and low risk scores in lung cancer patients compared to controls was 40% vs. 28% (high risk), and 16% vs. 22% (low risk), respectively (OR=1.93 (95% confidence interval of 1.3-2.9λ²=12.2, P=0.0005).

The frequency of high risk vs. neutral scores combined with low risk scores in lung cancer patients compared to controls was 40% vs. 28% (high risk), and 60% vs. 72% (neutral and low risk) respectively (OR=1.7 (95% confidence interval of 1.3-2.2, λ²=13.2, P=0.0003). In a 2×3 table of high, neutral and low scores for lung cancer and controls the frequencies were significantly different (λ²=14.7, P=0.007).

### Example 4

### 6 SNP Panel

This example presents an analysis of distributions of LCS scores derived for lung cancer sufferers and control resistant smokers using a 6 SNP panel as described below.

LCS scores for each subject were derived by assigning a score of +1 for the presence of each susceptibility genotype, or −1 for the presence of each protective genotype in the 6 SNP panel. The 6 SNP panel comprised the nAChR rs16969968 G/A polymorphism, the HHIP rs1489759 A/G polymorphism, the GYPA rs2202507 A/C polymorphism, the Solute Carrier Family 34 (SLC34A2) rs 2240997, the HLA-B associated transcript 3 (BAT3) rs 1052486 A/G polymorphism, and the C reactive protein (CRP) T/C rs 2808630 polymorphism. The scores were added to derive the 6 SNP panel LCS score for each subject. Table 15 below shows the distribution of LCS scores derived from the 6 SNP panel amongst the lung cancer patients and the resistant smoker controls.

TABLE 15
Lung cancer susceptibility score from the 6 SNP genotypes

| | Low risk score | | Neutral | High risk scores |
|---|---|---|---|---|
| Score | −2 or −3 | −1 | 0 | 1 or 2 or 3 |
| Controls | 46 | 86 | 230 | 124 |
| N = 486 | (9%) | (18%) | (47%) | (26%) |
| Lung cancer | 25 | 59 | 196 | 169 |
| N = 449 | (6%) | (13%) | (44%) | (38%) |

The frequency of high risk scores and low risk scores in lung cancer patients compared to controls was 38% vs. 26% (high risk), and 19% vs. 27% (low risk), respectively (OR=2.4 (95% confidence interval of 1.5-3.1λ²=17.5, P=0.00003).

The frequency of high risk vs. neutral scores combined with low risk scores in lung cancer patients compared to controls was 38% vs. 26% (high risk) and 62% vs. 74% (neutral and low risk) respectively (OR=1.8 (95% confidence interval of 1.3-2.4, λ²=16.0, P=0.00007). In a 2×3 table of high, neutral and low scores for lung cancer and controls the frequencies were significantly different (λ²=18.9, P=0.00008).

These data confirm that the combined presence of susceptibility genotypes and absence of protective genotypes allows a greater ability to discriminate between sufferers and controls, with more subjects being assigned a high risk or low risk LSC score.

### Example 5

### Substitution of a SNP in Linkage Disequilibrium

This example presents an analysis of distributions of LCS scores derived for lung cancer sufferers and control resistant smokers using a 4 SNP panel in which a SNP reported to be in LD is substituted for the original SNP, as described below.

Given the high concordance between the two nAChR SNPs (rs16969968 and rs1051730), the effect of substituting the former SNP with the latter in a 4 SNP panel using the same approach as described in Example 2 was assessed.

LCS scores for each subject were derived by assigning a score of +1 for the presence of each susceptibility genotype, or −1 for the presence of each protective genotype in the 6 SNP panel. The substituted 4 SNP panel comprised the nAChR rs1051730 C/T polymorphism, the HHIP rs1489759 A/G polymorphism, the GYPA rs2202507 A/C polymorphism, and the Solute Carrier Family 34 (SLC34A2) rs 2240997 polymorphisms. The scores were added to derive the substituted 4 SNP panel LCS score for each subject. Table 16 below shows the distribution of LCS scores derived from the substituted 4 SNP panel amongst the lung cancer patients and the resistant smoker controls.

TABLE 16
Lung cancer susceptibility score for the substituted 4 SNP panel

| | Low risk score | | Neutral | High risk scores |
|---|---|---|---|---|
| Score | −2 | −1 | 0 | 1 or 2 |
| Controls | 51 | 71 | 285 | 79 |
| N = 286 | (10%) | (15%) | (59%) | (17%) |
| Lung cancer | 24 | 56 | 249 | 120 |
| N = 449 | (5%) | (13%) | (56%) | (27%) |

As shown in Table 16 above, the scores did not change from the analysis reported in Example 2 above.

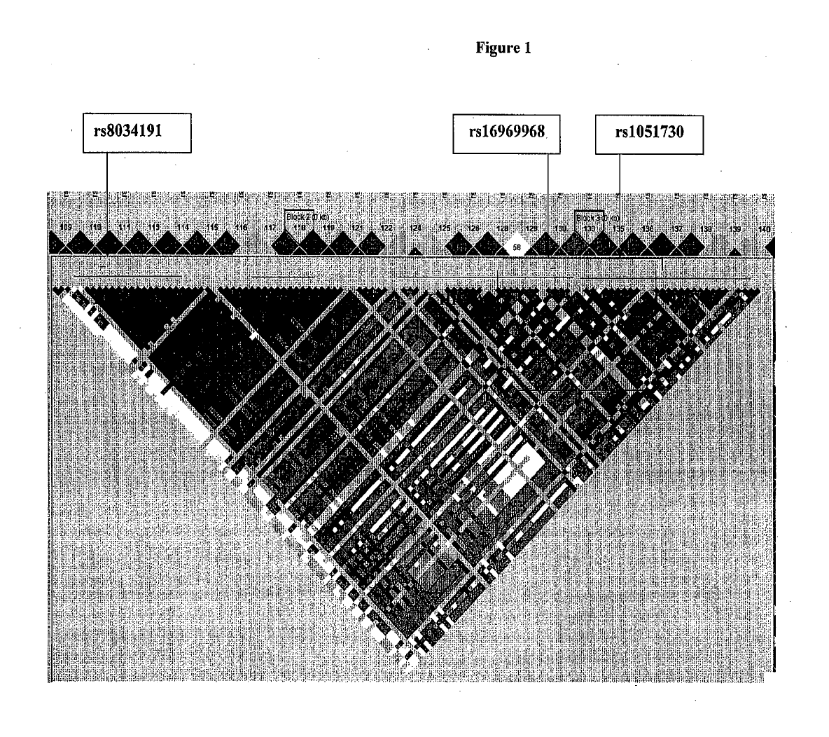

Table 17 below and FIG. 1 show the relationship between Nicotinic acetylcholine receptor polymorphisms in LD with the rs16969968 polymorphism, including the rs1051730 and rs8034191 polymorphisms. Complete LD between these 3 SNPs (D′=1.0) has been reported in HapMap.

TABLE 17
nAChR SNPs in LD

| | Major | Minor | Position | Closest Gene |
|---|---|---|---|---|
| rs8034191 | T | C | 76,593,078 | L0C123688 |
| | 0.567 | 0.433 | | (hypothetical) |
| rs16969968 | G | A | 76,669,980 | CHRNA5 |
| | 0.576 | 0.424 | | |
| rs1051730 | C | T | 76,681,394 | CHRNA3 |
| | 0.57 | 0.43 | | |

The rs8034191 polymorphism is a further example of a SNP in linkage disequilibrium with and with similar allele frequency to the rs16969968 SNP described herein.

In light of the analysis presented above, the degree of linkage disequilibrium, and the similarity in allele and genotype frequency, these 3 SNPs could readily be substituted for each other in a risk model or SNP panel.

### Example 6

Using the results of the univariate analysis above, nine risk genotypes were identified as either protective or susceptible (CHRNA 3/5 (rs16969968), BAT3 (rs1052486), CRR9/TERT (rs402710), HHIP (rs1489759), GYPA (rs2202507), FAM13A (rs 7671167), ADAM 19 (rs1422795), AGER (rs2070600), CRP (rs2808630). For each subject in the smoking control and lung cancer cohorts, the sum total of these SNP-based scores were added to the scores for the clinical variables to derive a total lung cancer susceptibility score. On FAR analysis, the plot of the total score with the frequency of lung cancer shows a linear relationship across quintiles (see FIG. 2). The distribution plot of the total scores according to control smokers and lung cancer cases is bimodal and the corresponding AUC is 0,70 for the 9 SNP panel used here. When the 12 most significant SNPs from a previous analysis was added to the 9 SNP panel, the AUC increases to 0.75.

### Example 7

Tables 18 to 24 below presents representative examples of polymorphisms in linkage disequilibrium with the polymorphisms specified herein. Examples of such polymorphisms can be located using public databases, such as that available at www.hapmap.org. Specified polymorphisms are shown in bold and parentheses. The rs numbers provided are identifiers unique to each polymorphism.

TABLE 18
nAChR polymorphisms in LD with rs16969968 (including rs1051730 and rs8034191).

| | | | | | |
|---|---|---|---|---|---|
| rs2869030 | rs12909921 | rs11858804 | rs11636131 | rs684513 | rs7178162 |
| rs4887053 | rs12910090 | rs11631834 | rs11637127 | rs7495275 | (rs1051730) |
| rs16969840 | rs12916396 | rs11631892 | rs11632604 | rs7165657 | rs8192481 |
| rs12439399 | rs12916558 | rs7497617 | rs12910289 | rs7166003 | rs3743078 |
| rs4436747 | rs2656071 | rs4887060 | rs7169751 | rs7178897 | rs3743077 |
| rs8043201 | rs2656069 | rs12593550 | rs1504546 | rs1472739 | rs1317286 |
| rs2869032 | rs2656068 | rs8026308 | rs16969931 | rs667282 | rs938682 |
| rs11856232 | rs2568496 | rs11636431 | rs12906951 | rs11636592 | rs12904589 |
| rs4381564 | rs2869048 | rs10450995 | rs3885951 | rs479385 | rs12914385 |
| rs2869045 | rs5020118 | rs10450964 | rs11633027 | rs16969948 | rs11637630 |
| rs2568495 | rs2017512 | rs965604 | rs931794 | rs588765 | rs2869546 |
| rs16969845 | rs2656065 | rs13180 | rs12913194 | rs6495306 | rs7177514 |
| rs2869046 | rs2568485 | rs2292116 | rs7180652 | rs16969949 | rs6495308 |
| rs2568498 | rs2568483 | rs9920411 | rs12916999 | rs12903839 | rs12443170 |
| rs12911087 | rs2656062 | rs2055588 | rs2036534 | rs17486278 | rs8042059 |
| rs2656057 | rs11639224 | rs3743079 | rs7164644 | rs1875869 | rs8042374 |
| rs1394371 | rs905742 | rs8033501 | rs12915366 | rs601079 | rs4887069 |
| rs12101964 | rs905741 | rs1062980 | rs12916483 | rs495956 | rs3743076 |
| rs12903150 | rs1964678 | rs17406522 | rs3813572 | rs680244 | rs3743075 |
| rs2656059 | rs2009746 | rs12441192 | rs3813571 | rs1878398 | rs3743074 |
| rs2656060 | rs2938674 | rs16969906 | rs3813570 | rs621849 | rs3743073 |
| rs2036530 | rs2938673 | rs3417 | rs12901682 | rs569207 | rs8040868 |
| rs12899425 | rs2958720 | rs11637193 | rs4886571 | rs637137 | rs8192475 |
| rs12899131 | rs1394372 | rs12914367 | rs4243083 | rs7180002 | rs1878399 |
| rs2568500 | rs17484235 | rs2055587 | rs2292117 | rs11633585 | rs6495309 |
| rs16969846 | rs17405883 | rs4362358 | rs11551779 | rs8026141 | rs1948 |
| rs2568484 | rs9972290 | rs5019044 | rs11858230 | rs692780 | rs7178270 |
| rs17483548 | rs4886569 | rs7171274 | rs8025429 | rs11637635 | rs3743072 |
| rs2869047 | rs3817092 | rs12906676 | rs4887062 | rs481134 | rs12914008 |
| rs17405217 | rs4299116 | rs6495304 | rs4887063 | rs951266 | rs17487223 |
| rs924840 | rs1504550 | rs7168796 | rs8053 | rs10519205 | rs950776 |
| rs2938671 | rs12591395 | rs16969914 | rs1979907 | rs555018 | rs17483721 |
| rs12910910 | rs9788682 | rs1979906 | rs647041 | rs2568487 | rs8043227 |
| rs9788721 | rs1979905 | rs12898919 | rs1847529 | rs7162301 | rs7164594 |
| rs4887064 | rs12903575 | rs1847528 | rs11634990 | rs16969920 | rs12907966 |
| rs17408276 | rs8041628 | rs11072766 | rs16969922 | rs1504547 | (rs16969968) |
| rs11630228 | rs11072767 | (rs8034191) | rs8024878 | rs518425 | rs2568488 |
| rs17484524 | rs4380026 | rs16969941 | rs11635346 | rs2656053 | rs8026728 |
| rs12591557 | rs880395 | rs514743 | rs2568491 | rs8042238 | rs10519203 |
| rs905740 | rs615470 | rs16969858 | rs8042260 | rs12914694 | rs7164030 |
| rs7163480 | rs2568492 | rs16969892 | rs7163730 | rs8037347 | rs12899226 |
| rs2656052 | rs8027404 | rs8031948 | rs7183333 | rs660652 | rs2568494 |
| rs11858961 | rs4461039 | rs4275821 | rs472054 | rs7181486 | rs12903295 |
| rs1504545 | rs7173512 | rs8029939 | rs2656073 | rs12904234 | rs952215 |
| rs4887065 | rs578776 | rs17483929 | rs7177092 | rs952216 | rs2036527 |
| rs6495307 | rs10519198 | rs16969899 | rs12902493 | rs11636732 | rs12910984 |
| rs2958719 | rs8032410 | rs11544874 | rs2944674 | rs8033506 | |

TABLE 19
HHIP polymorphisms in LD with rs1489759

| | | | | | |
|---|---|---|---|---|---|
| rs1032295 | rs2220516 | rs7655625 | rs9685759 | rs1032296 | rs2035743 |
| rs7673529 | rs7677662 | rs1032297 | rs6537292 | rs596165 | rs7700244 |
| rs1512281 | rs13104277 | rs451825 | rs6842331 | rs12504628 | rs10017175 |
| rs12641683 | rs1398243 | rs7697189 | rs6824927 | rs13118928 | rs7666523 |
| rs7681384 | rs12511230 | rs610411 | rs1186270 | rs11943195 | rs10028899 |
| rs12505157 | rs1542726 | rs4835637 | rs17019464 | rs426979 | rs4835638 |
| rs6820700 | rs404618 | rs1489757 | (rs1489759) | rs427260 | rs6829956 |
| rs383501 | rs17019485 | rs6854832 | rs386213 | rs6537296 | rs7340879 |
| rs1873297 | rs17019486 | rs11938704 | rs11932233 | rs462044 | rs3891822 |
| rs995759 | rs13140176 | rs1512285 | rs995758 | rs6828255 | rs6821114 |
| rs12509311 | rs1512288 | rs6845536 | rs4834988 | rs6817273 | rs1489762 |
| rs11100860 | rs593918 | rs1489761 | rs11934806 | rs2175586 | rs7692102 |
| rs6842889 | rs389937 | rs7673263 | rs6813222 | rs1473100 | rs7673872 |
| rs389291 | rs17019499 | rs7685166 | rs10519717 | rs13136959 | rs13147758 |
| rs9998537 | rs1844430 | rs13148031 | rs1828591 | rs6537297 | rs7689654 |
| rs6537295 | rs13126322 | rs720484 | rs6840009 | rs423625 | rs720485 |
| rs17019476 | rs13101284 | rs6811415 | rs6810579 | rs10013495 | rs6828540 |
| rs6816405 | rs13141641 | rs13113237 | rs17019477 | rs6852830 | rs2130339 |
| rs12510044 | rs2220548 | rs6830832 | rs457881 | rs12643826 | rs11938745 |
| rs6821908 | rs11724319 | rs6850426 | rs6829350 | rs1996020 | rs394216 |
| rs1489766 | rs11933312 | rs2130338 | rs1980057 | rs7670758 | rs11938808 |
| rs7671897 | rs7691995 | | | | |

TABLE 20
GYPA polymorpisms in LD with rs2202507

| | | | | | |
|---|---|---|---|---|---|
| rs13118083 | rs6849200 | rs885439 | rs11100855 | rs6814459 | rs6836202 |
| rs4835177 | rs7654571 | rs749316 | rs4533790 | rs1118190 | rs2657798 |
| rs7676032 | rs13142439 | rs13118515 | rs6856698 | rs989346 | rs4420930 |
| rs12510916 | rs13141892 | rs6828489 | rs6537279 | rs4376087 | rs1490146 |
| rs11100859 | rs13108250 | rs12645006 | rs12500355 | rs17019365 | rs13142879 |
| rs13137424 | rs12641258 | rs4835634 | rs6828795 | rs398962 | rs12640712 |
| rs1857835 | rs7654506 | rs1490147 | rs13149808 | rs6830386 | rs1505772 |
| rs990768 | rs11727645 | rs6825094 | rs12640763 | rs17766287 | rs951848 |
| rs11728562 | rs17712227 | rs7660767 | rs4371571 | rs1394999 | rs11731448 |
| rs1512287 | rs11100850 | rs4371572 | rs17767138 | rs1873296 | rs612550 |
| rs1505771 | rs7688932 | rs2719333 | rs6847170 | rs11722531 | rs7674433 |
| rs7683975 | rs2719332 | rs1490148 | rs461265 | rs4256191 | rs10009317 |
| rs4362772 | rs11940095 | rs13149519 | rs4321584 | rs2657799 | rs13109426 |
| rs4552414 | rs13143949 | rs1876116 | rs6537281 | rs17767210 | rs1505770 |
| rs13143967 | rs11100851 | rs7378179 | rs17019336 | rs1490149 | rs13144144 |
| rs2174527 | rs4240362 | rs17019340 | rs7689824 | rs13116441 | rs6842640 |
| rs6840917 | rs11726621 | rs2657805 | rs7684769 | rs6842885 | rs2657794 |
| rs4290852 | rs17019370 | rs7654708 | rs7655235 | rs4465995 | rs13117231 |
| rs390898 | rs12640256 | rs6836137 | rs986849 | rs13111832 | rs7675095 |
| rs973796 | rs7377575 | rs970022 | rs13135495 | rs2636153 | rs13116963 |
| rs4317155 | rs986241 | rs13135513 | rs13108069 | rs12641251 | rs4031150 |
| rs1505768 | rs13137063 | rs13108077 | rs12639777 | (rs2202507) | rs10029738 |
| rs13112056 | rs13113788 | rs1490150 | rs4306911 | rs10029931 | rs13117676 |
| rs13108244 | rs2048536 | rs6537278 | rs7681655 | rs1505762 | rs13108260 |
| rs12500946 | rs10030023 | rs4469023 | rs7675830 | rs438691 | rs8180243 |
| rs2657804 | rs4370082 | rs6537289 | rs443126 | rs11935246 | rs7661046 |
| rs7665807 | rs12512146 | rs625071 | rs6827794 | rs7375701 | rs4318599 |
| rs12499537 | rs438682 | rs1512282 | rs6852276 | rs1394998 | rs17019349 |
| rs423784 | rs10222998 | rs6858668 | rs988599 | rs11932998 | rs397724 |
| rs7671881 | rs13105210 | rs13121032 | rs612176 | rs17019376 | rs7654793 |
| rs2719341 | rs6537284 | rs627063 | rs11733975 | rs11727583 | rs6822064 |
| rs4642189 | rs7695767 | rs2719336 | rs13142776 | rs6840871 | rs4383570 |
| rs7678519 | rs6817612 | rs17019408 | rs2352767 | rs1505765 | rs7678522 |
| rs11735110 | rs7678427 | rs4493485 | rs4501169 | rs11726412 | rs1512289 |
| rs4266245 | rs7693416 | rs2719342 | rs2130499 | rs12503296 | rs7692044 |
| rs1907019 | rs440058 | rs17709487 | rs6811667 | rs2719340 | rs1512279 |
| rs7676787 | rs12645910 | rs2200942 | rs12499011 | rs987246 | rs1505766 |
| rs1602238 | rs17019381 | rs6834183 | rs6537285 | rs13103448 | rs1398245 |
| rs7699261 | rs6537286 | rs12499685 | rs11729536 | rs4292285 | rs4342151 |
| rs17019354 | rs17516 | rs17766168 | rs4610282 | rs2719337 | rs11722105 |

TABLE 21
SLC34A2 SNPs in LD with rs 2240997

| | | | | | |
|---|---|---|---|---|---|
| rs11731126 | rs10019851 | rs3796777 | rs10084927 | rs2240995 | rs2240996 |
| (rs2240997) | rs2240998 | | | | |

TABLE 22
CRP SNPs in LD with rs 2808630

| | | | | | |
|---|---|---|---|---|---|
| rs876538 | (rs2808630) | rs3093058 | rs3116651 | rs12760041 | rs11265259 |
| rs3116644 | rs12742963 | rs7553007 | rs4285692 | rs3122008 | rs3093069 |
| rs9628671 | rs3116650 | rs11588887 | rs2808628 | rs3122010 | rs9970836 |
| rs11265260 | rs6683589 | rs16842559 | rs3093080 | rs2027471 | rs4261114 |
| rs16842568 | rs1205 | rs12079772 | rs3116655 | rs2808629 | rs6413467 |
| rs1341665 | rs3122014 | rs7411419 | rs3116638 | rs3116656 | rs12727021 |
| rs6667499 | rs6413466 | rs12068753 | rs12081252 | rs3116649 | rs3116637 |
| rs2808634 | rs12081264 | rs2794520 | rs1130864 | rs2211321 | rs12081480 |
| rs3116648 | rs3093066 | rs2211322 | rs12569095 | rs3116647 | rs3093065 |
| rs2808635 | rs4275453 | rs3093079 | rs1800947 | rs13375877 | rs12728740 |
| rs3093077 | rs1417938 | rs13375891 | rs10437339 | rs3093075 | rs3093064 |
| rs12031749 | rs11265263 | rs3093073 | rs3093063 | rs16842596 | rs10437340 |
| rs3093072 | rs3122011 | rs3116653 | rs12083620 | rs3093071 | rs3093062 |
| rs16842599 | rs11265265 | rs3093070 | rs3093060 | rs3116652 | rs12049404 |

TABLE 23
ADAM19 SNPs in LD with rs1422795

| | | | | | |
|---|---|---|---|---|---|
| rs11739929 | rs4704869 | rs11466793 | rs4361500 | rs10054832 | rs6879450 |
| rs11466792 | rs4579243 | rs13436628 | rs9313606 | rs2287749 | rs4331881 |
| rs10065788 | rs9313607 | rs10063366 | rs4368711 | rs1609710 | rs9313608 |
| rs1833736 | rs4704870 | rs11466790 | rs11466763 | rs4704871 | rs7702683 |
| rs11466788 | rs2042247 | rs6869312 | rs10476052 | rs10054999 | rs3822692 |
| rs10054988 | rs3734029 | rs11466787 | rs11466762 | rs11466785 | rs11466760 |
| rs11466786 | rs11466761 | rs6885845 | rs11749762 | rs10035606 | rs1559146 |
| rs10071015 | rs11739062 | rs11744541 | rs6556068 | rs1990951 | rs13179607 |
| rs1990950 | rs3822695 | rs2161396 | rs11134764 | rs10039794 | rs10404 |
| rs2112690 | rs11466826 | rs10476058 | rs7732712 | rs11466784 | rs7721142 |
| rs11744671 | rs1559143 | rs10463021 | rs949800 | rs10044770 | rs4444952 |
| rs11746887 | rs11466825 | rs11466783 | rs7725295 | rs2287750 | rs12516927 |
| rs11466782 | rs10454970 | rs7714353 | rs11466822 | rs13357701 | rs11466818 |
| rs11742756 | rs11466821 | rs11744244 | rs11742401 | rs11466776 | rs4704744 |
| rs11466777 | rs10039535 | rs11741480 | rs11466817 | rs17657987 | rs3822585 |
| rs11750519 | rs6895849 | rs13354726 | rs9313632 | rs17054657 | rs9313633 |
| rs17054654 | rs10454971 | rs6893204 | rs6894959 | rs7717784 | rs13186619 |
| rs3844 | rs11466816 | rs2863747 | rs11749199 | rs11134775 | rs10475594 |
| rs2277027 | rs6866822 | rs11951889 | rs11466815 | rs17599812 | rs11466813 |
| rs6869994 | rs11466814 | rs10078178 | rs11750135 | rs17600807 | rs3822696 |
| rs10055180 | rs2902556 | rs10044656 | rs6861910 | rs13360140 | rs11134766 |
| rs10463022 | rs11466810 | rs868989 | rs3734032 | (rs1422795) | rs11465283 |
| rs868988 | rs6895343 | rs4704863 | rs11950414 | rs1559144 | rs12513538 |
| rs13173954 | rs12655736 | rs1422794 | rs11465282 | rs11134779 | rs1035434 |
| rs11134778 | rs11134799 | rs7709187 | rs10052412 | rs951958 | rs11466807 |
| rs1559145 | rs11466808 | rs10866659 | rs13353878 | rs4704872 | rs6866363 |
| rs7720584 | rs11954828 | rs4704864 | rs11466806 | rs11745505 | rs11466805 |
| rs4704742 | rs10475585 | rs10050985 | rs17054692 | rs6860540 | rs11739920 |
| rs7725846 | rs11746606 | rs11745566 | rs11466804 | rs10063083 | rs11466801 |
| rs10066571 | rs11466802 | rs17601035 | rs11466800 | rs11466766 | rs10058865 |
| rs4579242 | rs13155908 | rs10076407 | rs10067096 | rs4704867 | rs11466794 |
| rs12332707 | rs11134770 | rs3734031 | rs11740562 | rs6860507 | rs11134788 |
| rs6899205 | | | | | |

TABLE 24
FAM13A SNPs in LD with rs7671167

| | | | | | |
|---|---|---|---|---|---|
| rs1246642 | rs7697900 | rs10001420 | rs13119346 | rs13124770 | rs1246641 |
| rs17818123 | rs2670619 | rs2704592 | rs13148714 | rs2869966 | rs10033476 |
| rs6822256 | rs10516824 | rs11097214 | rs2869967 | rs7655875 | rs1708673 |
| rs2670630 | rs12640018 | rs2045517 | rs6835979 | rs6834414 | rs17014983 |
| rs12506327 | rs2609274 | rs13112207 | rs1795722 | rs1708674 | rs12646713 |
| rs1104633 | rs13112413 | rs11947489 | rs13150503 | rs2137715 | rs2464526 |
| rs13112464 | rs1708671 | rs1795724 | rs12649385 | rs6815270 | rs6844655 |
| rs17014931 | rs2670623 | rs6825998 | (rs7671167) | rs4507326 | rs1795721 |
| rs2704585 | rs2904264 | rs2013701 | rs2904262 | rs9992522 | rs1708676 |
| rs2869989 | rs2904259 | rs10007590 | rs7691517 | rs1795727 | rs6833401 |
| rs1903003 | rs9307061 | rs9993181 | rs6826407 | rs4342162 | rs1903004 |
| rs9991237 | rs17014934 | rs2464518 | rs11721751 | rs1458557 | rs10516827 |
| rs17014936 | rs1708678 | rs6843986 | rs1458558 | rs10516826 | rs12331870 |
| rs1795733 | rs1355838 | rs2609266 | rs2869972 | rs17014939 | rs1795731 |
| rs17015012 | rs2609268 | rs10516825 | rs6532094 | rs2670624 | rs6851538 |
| rs2446306 | rs2869971 | rs1795735 | rs11097210 | rs11725938 | rs1921679 |
| rs2904261 | rs1708670 | rs11097211 | rs4627822 | rs2178583 | rs7656238 |
| rs1708669 | rs16996151 | rs11097215 | rs2178584 | rs1921684 | rs1343921 |
| rs2670625 | rs13113298 | rs2178585 | rs4626161 | rs1807870 | rs2869984 |
| rs7691983 | rs13109988 | rs1961979 | rs10000140 | rs12504796 | rs7657630 |
| rs1903007 | rs6852288 | rs13109946 | rs12508893 | rs12502115 | rs13115960 |
| rs6852373 | rs11941615 | rs12508970 | rs2869987 | rs4555592 | rs6852928 |
| rs1708668 | rs8180333 | rs11934671 | rs2704577 | rs17014896 | rs17014952 |
| rs6857969 | rs11934674 | rs2609275 | rs6818212 | rs13140085 | rs13143981 |
| rs7440590 | rs11935197 | rs10015415 | rs2670618 | rs6532102 | rs11734924 |
| rs2085601 | rs6849143 | rs2458545 | rs938266 | rs11726708 | rs6838424 |
| rs6824116 | rs1588730 | rs5026462 | rs6828135 | rs2704573 | rs12504536 |
| rs7666393 | rs4390994 | rs3931352 | rs16996143 | rs17014898 | rs1398937 |
| rs2869990 | rs17015025 | rs17817631 | rs1795739 | rs17014962 | rs6845151 |
| rs17015027 | rs16996144 | rs1398942 | rs17014963 | rs9790655 | rs2280099 |
| rs11737182 | rs1795738 | rs1513808 | rs1533288 | rs8582 | rs11737260 |
| rs12505696 | rs2704571 | rs938265 | rs12645173 | rs9307054 | rs1795737 |
| rs1513807 | rs1996139 | rs17821105 | rs1921687 | rs17014901 | rs1708684 |
| rs6816472 | rs3733448 | rs7660885 | rs1795734 | rs13141671 | rs6835031 |
| rs938269 | rs9307055 | rs2670620 | rs17768938 | rs1513811 | rs938268 |
| rs10470936 | rs12509305 | rs17014966 | rs6842150 | rs938267 | rs10028121 |
| rs1795740 | rs1533291 | rs874147 | rs1533290 | rs11945054 | rs1398941 |
| rs1513822 | rs6856010 | rs10433949 | rs9307059 | rs1398940 | rs17014977 |
| rs756175 | rs1554003 | rs1921681 | rs1398939 | rs6818976 | rs12639677 |
| rs10433881 | rs7686954 | rs1398938 | rs2670626 | rs756176 | rs13139223 |
| rs1921682 | rs12508524 | rs2670629 | rs7682131 | rs13138927 | rs10033484 |
| rs1708661 | rs13118939 | rs11725475 | rs7669140 | rs7697075 | rs4352442 |
| rs13119345 | rs10004795 | | | | |

### DISCUSSION

The above results show that several polymorphisms were associated with either increased or decreased risk of developing lung cancer. The associations of individual polymorphisms on their own, while of discriminatory value, are unlikely to offer an acceptable prediction of disease. However, in combination these polymorphisms distinguish susceptible subjects from those who are resistant (for example, between the smokers who develop lung cancer and those with the least risk with comparable smoking exposure). The polymorphisms represent exonic polymorphisms known to alter amino-acid sequence (and likely expression and/or function) in a number of genes involved in processes known to underlie lung remodelling and lung cancer, and in one case a silent mutation having no effect on amino acid composition. The polymorphisms identified here are found in genes encoding proteins central to these processes which include inflammation, matrix remodelling, oxidant stress, DNA repair, cell replication and apoptosis.

In the comparison of smokers with lung cancer and matched smokers with near normal lung function (lowest risk for lung cancer despite smoking), several polymorphisms were identified as being found in significantly greater or lesser frequency than in the comparator groups (sometimes including the blood donor cohort). Due to the small cohort of lung cancer patients, polymorphisms where there are only trends towards differences (P=0.06-0.25) may be included in the analyses, although in the combined analyses only those polymorphisms with the most significant differences were utilised.
In the analysis of the nAChR rs16969968 G/A polymorphism, the AA genotype was found to be significantly greater in the lung cancer cohort compared to the resistant smoker cohort (OR=1.8, P=0.005), consistent with a susceptibility role (see Table 1). The A allele was found to be significantly greater in the lung cancer cohort compared to the resistant smoker cohort (OR=1.4, P=0.001), consistent with a susceptibility role.
In the analysis of the nAChR rs1051730 C/T polymorphism, the TT genotype was found to be significantly greater in the lung cancer cohort compared to the resistant smoker cohort (OR=1.9, P=0.002), consistent with a susceptibility role (see Table 2). The T allele was found to be significantly greater in the lung cancer cohort compared to the resistant smoker cohort (OR=1.4, P=0.0005), again consistent with a susceptibility role.
In the analysis of the HHIP rs1489759 A/G polymorphism, the GG genotype was found to be greater in the resistant smoker controls compared to the lung cancer cohort (OR=0.70, P=0.05), consistent with a protective role (see Table 3).
In the analysis of the GYPA rs2202507 A/C polymorphism, the CC genotype was found to be greater in the resistant smoker controls compared to the lung cancer cohort (OR=0.70, P=0.02), consistent with a protective role (see Table 4).
In the analysis of the SLC34A2 rs 2240997 polymorphism, the GA and AA genotypes were found to be greater in the lung cancer cohort compared to the resistant smoker cohort (OR=1.53, P=0.009) consistent with each having a susceptibility role. The A allele was found to be significantly greater in the lung cancer cohort compared to the resistant smoker controls (OR=1.4, P=0.01), consistent with a susceptibility role (see Table 5).
In the analysis of the BAT3 rs 1052486 polymorphism, the GG genotype was found to be significantly greater in the lung cancer cohort compared to the resistant smoker cohort (OR=1.4, P=0.08), consistent with a susceptibility role (see Table 6). The G allele was found to be significantly greater in the lung cancer cohort compared to the resistant smoker controls (OR=1.2, P=0.07), consistent with a susceptibility role (see Table 6). Stratification of the lung cancer cohort by available spirometric data (n=412) into those with and without COPD (according to GOLD ≧2 criteria) identified the association of the GG genotype with the lung cancer+COPD phenotype (23% in controls vs. 31% in LC+COPD, OR=1.50, P=0.03). The GG genotype was significantly greater in the lung cancer with COPD group than the lung cancer only group (31% vs. 21%, OR=1.68, P=0.02) The GG genotype of the BAT3 SNP appears to confer susceptibility for lung cancer in those with COPD (Table 6).
In the analysis of the CRP T/C rs 2808630 polymorphism, the GG genotype was found to be greater in the smoker controls compared to the lung cancer cohort (OR=0.68, P=0.09), consistent with a protective role (see Table 7). After stratification of the lung cancer cohort by available spirometric data (n=409) into those with and without COPD (according to GOLD ≧2 criteria) a significant association of the CC genotype with the lung cancer only group was identified (11% in controls vs. 5%, OR 0.47, P=0.02). The frequency of the CC genotype was significantly lower in the lung cancer only cohort compared to lung cancer with COPD (5% vs. 9%, OR=0.54, P=0.03). This indicates that the CC genotype of the CRP SNP was associated with susceptibility to lung cancer in those without COPD (Table 7A).
In the analysis of the CRR9 (rs 402710) polymorphism, the GG genotype was comparable between lung cancer cases compared to controls (47% vs. 44%, OR=1.10, P=0.45) (Table 8). When the lung cancer cases were divided according to their spirometry (n=422) into those with COPD and without COPD (according to GOLD ≧2 criteria), the frequency of the GG genotype was 42% in lung cancer with COPD (vs. 44% in controls, OR=0.90, P=0.54) and 53% in lung cancer only subjects (vs. 44% in controls, OR=1.40, P=0.05) respectively (Table 8). The GG genotype is raised in the lung cancer only patients compared to the lung cancer with COPD group (53% vs. 42%, OR=1.54, P=0.03). The GG genotype of the TERT/CRR9SNP confers susceptibility for lung cancer (Table 8). Identical results were obtained when the CRR9 rs402710 polymorphism, reported to be in LD with the rs401681 polymorphism, was independently analysed.
The frequency of the CC genotype of the ADAM19 (rs 1422795) SNP was mildly reduced in the controls compared to the lung cancer group (9% vs. 13%, OR=1.44, P=0.08) (Table 9). When the lung cancer cases were divided according to their spirometry (n=421) into those with COPD and without COPD (according to GOLD ≧2 criteria) the effect size of the CC genotype remained the same, compared to controls (lung cancer with COPD 13%, OR=1.51, P=0.10 and lung cancer without COPD 13%, OR=1.40, P=0.20), although p-values were degraded due to smaller numbers. When the CC genotype frequency of the controls is compared to those with COPD and lung cancer (9% vs. 13%, OR=1.48, P=0.05) the larger cohort identifies a significant increase in the CC genotype in those with the susceptible phenotype. The CC genotype thus confers susceptibility to lung cancer in those with COPD (Table 9).
In the analysis of the FAM13A rs7671167 polymorphism, the CC genotype was significantly increased in smoking controls (30%) compared to the lung cancer cohort (21%, OR=0.64, P=0.003), consistent with a protection role (see Table 10).
In the analysis of the BICD1 rs161974 C/T polymorphism, the C allele was significantly significantly increased in lung cancer sufferers (63%) compared to the resistant smoker cohort (58%, OR=1.24, P=0.022), consistent with a susceptibility role (see Table 11).
In the analysis of the BICD1 rs2630578 C/G polymorphism, the CC genotype was significantly increased in lung cancer sufferers (6%) compared to the resistant smoker cohort (3%, OR=1.80, P=0.067), consistent with a susceptibility role (see Table 12).
When compared to all smoking controls, the CC genotype was significantly increased in lung cancer sufferers (OR=2.26, P=0.004), confirming a susceptibility role.

It is accepted that the disposition to lung cancer is the result of the combined effects of the individual's genetic makeup and other factors, including their lifetime exposure to various aero-pollutants including tobacco smoke. Similarly it is accepted that lung cancer encompasses several obstructive lung diseases and characterised by impaired expiratory flow rates (e.g. FEV1). The data herein suggest that several genes can contribute to the development of lung cancer. A number of genetic mutations working in combination either promoting or protecting the lungs from damage are likely to be involved in elevated resistance or susceptibility to lung cancer.

In one embodiment, from the analyses of the individual polymorphisms 5 protective genotype and 9 susceptibility genotypes were identified and analysed for their frequencies in the smoker cohort consisting of resistant smokers and those with lung cancer. A SNP score was determined for each subject by assigning a score of +1 for the presence of a susceptibility genotype and −1 for the presence of a protective genotype. These scores were added to derive a SNP score for each subject.

The frequency of high risk LCS scores and low risk LCS scores in resistant smokers and smokers with lung cancer were compared according to the LCS score derived from a 4 SNP panel consisting of the SNPs identified in Example 2 herein. The frequency of high risk 4 SNP panel LCS scores was 27% amongst lung cancer sufferers, compared to 17% in resistant smokers. Conversely, the frequency of low risk 4 SNP panel LCS scores was 18% amongst lung cancer sufferers, compared to 25% in resistant smokers.

The frequency of high risk LCS scores and low risk LCS scores in resistant smokers and smokers with lung cancer were compared according to the LCS score derived from a 5 SNP panel consisting of the SNPs identified in Example 3 herein. The frequency of high risk 5 SNP panel LCS scores was 40% amongst lung cancer sufferers, compared to 28% in resistant smokers. Conversely, the frequency of low risk 5 SNP panel LCS scores was 16% amongst lung cancer sufferers, compared to 22% in resistant smokers.

When the frequency of high risk LCS scores and low risk LCS scores in resistant smokers and smokers with lung cancer were compared according to the LCS score derived from a 6 SNP panel consisting of the SNPs identified in Example 4 herein, the frequency of high risk 6 SNP panel LCS scores was 38% amongst lung cancer sufferers, compared to 26% in resistant smokers. Conversely, the frequency of low risk 6 SNP panel LCS scores was 19% amongst lung cancer sufferers, compared to 27% in resistant smokers.

These findings indicate that the methods of the present invention may be predictive of lung cancer in an individual well before symptoms present.

Importantly, a comparison of the frequencies of high, neutral, and low risk scores generated with the 4 SNP panel compared to the 6 SNP panel shows that the 6 SNP panel identifies a larger subgroup of control smokers who are at low or neutral risk. This has important implications in rationing or prioritising medical interventions.

These findings indicate that the methods of the present invention may be used to identify subsets of nominally at risk individuals (and particularly smokers) who are at low to average risk of lung cancer, and are thus not suitable for an intervention.

These findings therefore also present opportunities for therapeutic interventions and/or treatment regimens, as discussed herein. Briefly, such interventions or regimens can include the provision to the subject of motivation to implement a lifestyle change, or therapeutic methods directed at normalising aberrant gene expression or gene product function. In another example, a given susceptibility genotype is associated with increased expression of a gene relative to that observed with the protective genotype. A suitable therapy in subjects known to possess the susceptibility genotype is the administration of an agent capable of reducing expression of the gene, for example using antisense or RNAi methods. An alternative suitable therapy can be the administration to such a subject of an inhibitor of the gene product. In still another example, a susceptibility genotype present in the promoter of a gene is associated with increased binding of a repressor protein and decreased transcription of the gene. A suitable therapy is the administration of an agent capable of decreasing the level of repressor and/or preventing binding of the repressor, thereby alleviating its downregulatory effect on transcription. An alternative therapy can include gene therapy, for example the introduction of at least one additional copy of the gene having a reduced affinity for repressor binding (for example, a gene copy having a protective genotype).

Suitable methods and agents for use in such therapy are well known in the art, and are discussed herein.

The identification of both susceptibility and protective polymorphisms as described herein also provides the opportunity to screen candidate compounds to assess their efficacy in methods of prophylactic and/or therapeutic treatment. Such screening methods involve identifying which of a range of candidate compounds have the ability to reverse or counteract a genotypic or phenotypic effect of a susceptibility polymorphism, or the ability to mimic or replicate a genotypic or phenotypic effect of a protective polymorphism.

Still further, methods for assessing the likely responsiveness of a subject to an available prophylactic or therapeutic approach are provided. Such methods have particular application where the available treatment approach involves restoring the physiologically active concentration of a product of an expressed gene from either an excess or deficit to be within a range which is normal for the age and sex of the subject. In such cases, the method comprises the detection of the presence or absence of a susceptibility polymorphism which when present either upregulates or downregulates expression of the gene such that a state of such excess or deficit is the outcome, with those subjects in which the polymorphism is present being likely responders to treatment.

### INDUSTRIAL APPLICATION

The present invention is directed to methods for assessing a subject's risk of developing lung cancer. The methods comprise the analysis of polymorphisms herein shown to be associated with increased or decreased risk of developing lung cancer, or the analysis of results obtained from such an analysis. The use of polymorphisms herein shown to be associated with increased or decreased risk of developing lung cancer in the assessment of a subject's risk are also provided, as are nucleotide probes and primers, kits, and microarrays suitable for such assessment. Methods of treating subjects having the polymorphisms herein described are also provided. Methods for screening for compounds able to modulate the expression of genes associated with the polymorphisms herein described are also provided.

### PUBLICATIONS

Alberg A J, Samet J M. Epidemiology of lung cancer. Chest 2003, 123, 21s-49s.
Anthonisen N R. Prognosis in COPD: results from multi-center clinical trials. Am Rev Respir Dis 1989, 140, s95-s99.
Kuller L H, et al. Relation of forced expiratory volume in one second to lung cancer mortality in the MRFIT. Am J Epidmiol 1190, 132, 265-274.
Mayne S T, et al. Previous lung disease and risk of lung cancer among men and women nonsmokers. Am J Epidemiol 1999, 149, 13-20.
Nomura a, et al. Prospective study of pulmonary function and lung cancer. Am Rev Respir Dis 1991, 144, 307-311.
Schwartz A G. Genetic predisposition to lung cancer. Chest 2004, 125, 86s-89s.
Skillrud D M, et al. Higher risk of lung cancer in COPD: a prospective matched controlled study. Ann Int Med 1986, 105, 503-507.
Tockman M S, et al. Airways obstruction and the risk for lung cancer. Ann Int Med 1987, 106, 512-518.
Wu X, Zhao H, Suk R, Christiani D C. Genetic susceptibility to tobacco-related cancer. Oncogene 2004, 23, 6500-6523.

All patents, publications, scientific articles, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents.

The specific methods and compositions described herein are representative of various embodiments or preferred embodiments and are exemplary only and not intended as limitations on the scope of the invention. Other objects, aspects, examples and embodiments will occur to those skilled in the art upon consideration of this specification, and are encompassed within the spirit of the invention as defined by the scope of the claims. It will be readily apparent to one skilled in the art that varying substitutions and modifications can be made to the invention disclosed herein without departing from the scope and spirit of the invention. The invention illustratively described herein suitably can be practiced in the absence of any element or elements, or limitation or limitations, which is not specifically disclosed herein as essential. Thus, for example, in each instance herein, in embodiments or examples of the present invention, any of the terms “comprising”, “consisting essentially of”, and “consisting of” may be replaced with either of the other two terms in the specification, thus indicating additional examples, having different scope, of various alternative embodiments of the invention. Also, the terms “comprising”, “including”, containing”, etc. are to be read expansively and without limitation. The methods and processes illustratively described herein suitably may be practiced in differing orders of steps, and that they are not necessarily restricted to the orders of steps indicated herein or in the claims. It is also that as used herein and in the appended claims, the singular forms “a,” “an,” and “the” include plural reference unless the context clearly dictates otherwise. Thus, for example, a reference to “a host cell” includes a plurality (for example, a culture or population) of such host cells, and so forth. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as described in the following indicative claims.

1. A method of determining a subject's risk of developing lung cancer comprising analysing a sample from said subject for the presence or absence of one or more polymorphisms selected from the group consisting of:
rs1489759 A/G in the gene encoding Hedgehog Interacting Protein (HHIP);
rs2240997 G/A in the gene encoding Solute Carrier Family 34 (SLC34A2);
rs7671167 T/C in the Family with sequence similarity 13A (FAM13A) gene;
rs161974 C/T in gene encoding Bicaudal D homologue 1 (BICD1);
rs2630578 C/G in gene encoding BICD1; or
one or more polymorphisms in linkage disequilibrium with one or more of said polymorphisms, wherein the presence or absence of said polymorphism is indicative of the subject's risk of developing lung cancer.


SEQUENCE LISTING (9 sequences)

SEQ ID NO: 1 - DNA, 51 bp, Artificial
  Synthetic - sequence on which polymorphism-specific oligonucleotide(s) were designed
tagaaacacattggaagctgcgctcrattctattcgctacattacaagaca

SEQ ID NO: 2 - DNA, 51 bp, Artificial
  Synthetic - sequence on which polymorphism-specific oligonucleotide(s) were designed
agacgacactagtttttaaagttttkattaatcgctgctgtgaagctgcat

SEQ ID NO: 3 - DNA, 51 bp, Artificial
  Synthetic - sequence on which polymorphism-specific oligonucleotide(s) were designed
gaaattgttttctttggacaacttgrcaaaaaccaatcatctgtcagtgat

SEQ ID NO: 4 - DNA, 51 bp, Artificial
  Synthetic - sequence on which polymorphism-specific oligonucleotide(s) were designed
aggccagaggctgtctaccagactaygtatagtaagatgcaagcaactgaa

SEQ ID NO: 5 - DNA, 51 bp, Artificial
  Synthetic - sequence on which polymorphism-specific oligonucleotide(s) were designed
caggagttcatatctagagagctgtragtcaggccttccttcttagcgggt

SEQ ID NO: 6 - DNA, 51 bp, Artificial
  Synthetic - sequence on which polymorphism-specific oligonucleotide(s) were designed
agcagttgtacttgatgtcgtgtttrtagcctggggctttgatgatggccc

SEQ ID NO: 7 - DNA, 51 bp, Artificial
  Synthetic - sequence on which polymorphism-specific oligonucleotide(s) were designed
gtgatggtgggagaagccacaccagrccctccagcccctggccctgcaggc

SEQ ID NO: 8 - DNA, 51 bp, Artificial
  Synthetic - sequence on which polymorphism-specific oligonucleotide(s) were designed
tgggcaagcagcttgcgcctccaacygagaaaggaccagagggtagaatat

SEQ ID NO: 9 - DNA, 51 bp, Artificial
  Synthetic - sequence on which polymorphism-specific oligonucleotide(s) were designed
cattaagaaagaattaggtaaattcyaaaacataagggaatactatgacaa